(19) United States
(12) Patent Application Publication
(10) Pub. No.: US 2017/0136127 A1
(43) Pub. Date: May 18, 2017

(54) HYDROGELS FOR TREATING AND AMELIORATING CANCERS AND POTENTIATING THE IMMUNE SYSTEM AND METHODS OF MAKING AND USING THEM

(71) Applicant: Vicus Therapeutics, LLC, Morristown, NJ (US)

(72) Inventors: John MAKI, Mendham, NJ (US); Newell BASCOMB, Hendersonville, NC (US); Fredric YOUNG, Los Altos, CA (US)

(21) Appl. No.: 15/320,732

(22) PCT Filed: Jul. 1, 2015
(86) PCT No.: PCT/US15/38849

§ 371 Date: Dec. 20, 2016

Related U.S. Application Data
(60) Provisional application No. 62/019,799, filed on Jul. 1, 2014.

(51) Int. Cl. A61K 47/42; A61K 31/138; A61K 31/407; A61K 38/20; A61K 9/00

(52) U.S. Cl. CPC A61K 47/42; A61K 38/2013; A61K 9/0024; A61K 31/407; A61K 31/138

(57) ABSTRACT

In alternative embodiments, provided are pharmaceutical compositions, formulations, kits and other products of manufacture, comprising a sterile hydrogel comprising a hydrogel material and active ingredients including one or a plurality of compositions or compounds, which may comprise: a biologic, a drug or an immunostimulating agent or reagent; an antigen or an immunogen, or a plurality of antigens or immunogens; an anticancer agent or reagent, or any combination thereof.

### RELATED APPLICATIONS

This patent Convention Treaty (PCT) International application claims the benefit of priority under 35 U.S.C. §119(e) of U.S. Provisional Application Ser. No. 62/019,799, filed Jul. 1, 2014. The aforementioned application is expressly incorporated herein by reference their entirety and for all purposes.

### FIELD OF THE INVENTION

This invention relates generally to medicine, pharmaceutical formulations and medical devices. In alternative embodiments, provided are pharmaceutical compositions, formulations, kits and other products of manufacture, comprising a sterile hydrogel comprising a hydrogel material and active ingredients including one or a plurality of compositions or compounds, which may comprise: a biologic, a drug or an immunostimulating agent or reagent; an antigen or an immunogen, or a plurality of antigens or immunogens; an anticancer agent or reagent, or any combination thereof.

### BACKGROUND

Therapeutic cancer vaccines have been approved by the FDA and a diverse range of therapeutic cancer vaccines directed against a spectrum of tumor-associated antigens are currently being evaluated in clinical trials. However, the tumor microenvironment and other immunosuppressive entities can potentially limit the efficacy of vaccines, and producing effective treatment vaccines has proven much more difficult and challenging than developing cancer preventive vaccines. To be effective, cancer treatment vaccines must achieve two goals. Like traditional vaccines and cancer preventive vaccines, cancer treatment vaccines must stimulate specific immune responses against the correct target. The immune responses must be powerful enough to overcome the barriers that cancer cells use to protect themselves from attack by B cells and killer T cells. A variety of approaches have been tried to counteract this, for example, vaccines combined with drugs or cancer therapies, e.g., as immune checkpoint inhibitors, chemotherapeutics and/or radiation, are being evaluated both in preclinical and clinical studies. New approaches in cancer immunotherapeutics are needed to address these challenges.

Chemotherapy also is important in cancer treatment, but chemotherapy drugs act by damaging high proliferating cells, and damage to normal cells results in chemotherapy toxicities and side effects. Chemotoxicity can be seen most in actively dividing tissues such bone marrow, hair follicles and gastrointestinal mucosa. New approaches in cancer chemotherapeutics are needed to address these challenges.

### SUMMARY

In alternative embodiments, provided are products of manufacture, devices or compositions, comprising:

(a) a sterile hydrogel comprising a hydrogel material, wherein the hydrogel is:
(i) in a substantially liquid form capable of setting, gelling or self-assembling;
(ii) a partially assembled or gelled hydrogel, in a partially assembled or gelled form; or,
(iii) in a set, gelled or self-assembled state; or a substantially set, gelled or self-assembled state, and optionally the set, gelled or self-assembled state is in situ; and

(b) one or a plurality of compositions or compounds comprising:
(i) a biologic, a drug or an immunostimulating agent or reagent,
(ii) an antigen or an immunogen, or a plurality of antigens or immunogens,
(iii) (1) a biologic, a drug or an immunostimulating agent or reagent, and
(2) an antigen or an immunogen, or a plurality of antigens or immunogens,
(iv) an anticancer agent or reagent, or
(v) any combination of (i), (ii), (iii) and (iv), or all of (i) to (iv).

In alternative embodiments:

(a) the sterile hydrogel material or sterile hydrogel is mixed with the one or the plurality of compositions or compounds provided herein;

(b) the one or the plurality of compositions or compounds provided herein are first mixed in a sterile pure water or a sterile isotonic solution or buffer; or

(c) the one or the plurality of compositions or compounds provided herein are mixed with the sterile hydrogel material or sterile hydrogel:
(i) while the hydrogel is still in a substantially liquid state, un-self-assembled state, or ungelled state;
(ii) before the hydrogel has self-assembled, set or gelled,
(iii) before the hydrogel has set or self-assembled into a 3D hydrogel,
(iv) after the set, gelled or self-assembled hydrogel, or the substantially set, gelled or self-assembled hydrogel, has been disrupted or sheared; or
(v) at the same time the hydrogel has set, gelled or self-assembled hydrogel, or the hydrogel has substantially set, gelled or self-assembled.

In alternative embodiments:

(a) the hydrogel is capable of self-assembling, gelling or setting when exposed to an environment comprising a salt concentrations ≧1 mM (or gelation, self-assembly or setting is initiated by salt concentrations ≧1 mM);

(b) the hydrogel is capable of self-assembling, gelling or setting into a 3D hydrogel having a nanometer scale and/or a fibrous structure with an average pore size of between about 50 to 200 nm; or

(c) the hydrogel is at a concentration of about: 0.1% to 5% (w/v), 0.5% to 4% (w/v), 1% to 3% (w/v), 1% to 10% (w/v), 1% to 15% (w/v), 1% to 20% (w/v), 1% to 25% (w/v), 1% to 30% (w/v), 1% to 40% (w/v), or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more (w/v).

In alternative embodiments:

(a) the hydrogel or hydrogel material comprises a self-assembling peptide;

(b) the hydrogel or hydrogel material comprises a plurality of synthetic peptides characterized by stable B-sheet structure with ionic side-chain interactions after setting, gelling or self-assembling;

(c) the hydrogel or hydrogel material comprises a 16-amino acid synthetic peptide (Ac-[RADA]₄-CONH₂), or SEQ ID NO:1, and optionally the hydrogel comprises PURAMATRIX™ (PuraMatrix™) (BD Biosciences, San Jose, Calif.), or PURADERM™ (PuraDerm™) (3DMatrix, Ltd, Tokyo, Japan);

(d) the hydrogel or hydrogel material comprises a self-assembling peptide comprising the sequence Lys-Leu-Asp-Leu-Lys-Leu-Asp-Leu-Lys-Leu-Asp-Leu (KLDL)₃ (SEQ ID NO:2);

(e) the hydrogel or hydrogel material comprises a self-assembling peptide comprising the sequence Ile-Glu-Ile-Lys-Ile-Glu-Ile-Lys-Ile-Glu-Ile-Lys-Ile (IEIK)₃I (SEQ ID NO:3);

(f) the hydrogel or hydrogel material comprises a cellulose, a chitin, a chitosan or a deacetylated chitin, a laminin, a collagen, an elastin, a fibrin, a gelatin, an alginic acid, a hyaluronic acid (HA), or a combination thereof,

wherein optionally the HA comprise a thiolated HA or a tyraminated HA;

or optionally the collagen comprises a collagen IV or a collagen I,

or optionally the cellulose comprises a hemicellulose methyl cellulose (MC), a hydroxypropyl cellulose (HPC), a hydroxypropylmethyl cellulose (HPMC), a carboxymethyl cellulose (CMC) or a cellulose-inorganic hybrid hydrogel;

(g) the hydrogel or hydrogel material comprises a polyethylene glycol (PEG), a polyethelene glycol diacrylate (PEGDA), an ethylene glycol dimethacrylate (EGDMA); a cyclodextrin; a p-dioxanone; a hydroxyethyl methacrylate; a poly(methyl methacrylate); a methylene-bis-acrylamide; a poly(acrylic acid); a polyacrylonitrile; a poly(butylene oxide); a polycaprolactone; a poly(ethylene imine); a poly(ethylene oxide); a poly(ethyl methacrylate); a propylene fumarate; a poly(glucosylethyl methacrylate); a poly(hydroxy butyrate); a poly(hydroxyethyl methacrylate); a poly(hydroxypropyl methacrylamide); a poly(lactic acid); a poly(lactic-co-glycolic acid); PNIPAAm, poly(N-isopropyl acrylamide); a poly(N-vinyl pyrrolidone); a polypropylene oxide); a poly(vinyl alcohol); a poly(vinyl acetate); a poly(vinyl amine), or any combination thereof; or

(h) the hydrogel or hydrogel material comprises any combination of (a) to (g).

In alternative embodiments:

(a) the sterile pure water or a sterile isotonic solution or buffer comprises a saline, a phosphate buffered saline (PBS), or an equivalent buffer;

(b) the product of manufacture, device or composition of (a), wherein: (1) the saline is used at an undiluted concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or, (2) the PBS is at a concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more.

In alternative embodiments, the antigen, immunogen, or a plurality of antigens or immunogens, comprises:

(a) a synthetic, recombinant, partially purified, substantially purified or purified antigen, immunogen, or a plurality of antigens or immunogens, or any combination thereof;

(b) a small molecule or a biological molecule, wherein optionally the biological molecule is or comprises a peptide, a polypeptide, a carbohydrate, a lipid, or any combination thereof, and optionally the polypeptide comprises an antibody, or an anti-cancer or anti-tumor antibody, and optionally the anti-cancer or anti-tumor antibody is an alemtuzumab, a brentuximab vedotin, a cetuximab, a gemtuzumab ozogamicin, an abritumomab tiuxetan, a nimotuzumab, an ofatumumab, a panitumumab, a rituximab, a tositumomab, or a trastuzumab;

(c) a cancer or tumor antigen, immunogen, or a plurality of antigens or immunogens, or any combination thereof;

(d) a cancer or a tumor cell extract, or a processed cancer or tumor cell, wherein optionally the processed cancer or tumor cell is a minced cancer or tumor tissue or cell, and optionally the cancer or tumor tissue is minced with a device for making a mixed thickness skin micrograft or a split-thickness skin graft, or an XPANSION® device or an XPANSION MICROGRAFTING SYSTEM® (SteadMed Medical, Fort Worth, Tex.), and optionally the processed cancer or tumor cell is an irradiated cancer or tumor cell;

(e) the antigen, immunogen, or plurality of antigens or immunogens of any of (a) to (d), wherein the antigen, immunogen, or plurality of antigens or immunogens is mixed or treated with a cross-linking agent, a glutaraldehyde, a formaldehyde, a preservative, a neomycin, a polymyxin B, polihexanide, or any combination thereof, or the antigen, immunogen, or plurality of antigens or immunogens are irradiated; or

(f) any combination of (a) through (e).

In alternative embodiments: the biologic, a drug or an immunostimulating agent or reagent comprises:

(a) a cytokine, wherein optionally the cytokine comprises an IL-2 or an interferon (IFN), and optionally the interferon is an alpha-IFN or a gamma-IFN; and optionally the IL-2 is a recombinant IL-2, an aldesleukin, or a PROLEUKIN (Prometheus Laboratories), wherein optionally the IL-2, recombinant IL-2, or aldesleukin is dosages at about: 0.1 to 20, 1.0 to 20, 1 to 10, or 4 to 5, or 4.5 millions of IUs per cycle; or is dosaged for: 1 to 5, 2 to 4, or 3 cycles number of cycles of therapy;

(b) an immune checkpoint blockade agent, or an agent that blocks the interaction between a transmembrane programmed cell death 1 protein (PD-1; also known as CD279) and its ligand, PD-1 ligand 1 (PD-L1), or an ipilumumab (CTLA-4 mAb) or nivolumab (PD-1 mAb), or pembrolizumab (PD-1 mAb), or a lambrolizumab (a PD-L1 mAb);

(c) an activator of a pattern recognition receptor (PRR) or a toll-like receptor 7 (TLR7), or an imiquimod;

(d) chemotherapeutic agent, wherein optionally the chemotherapeutic agent comprises a doxorubicin or a carboplatin, or comprises an inducer of apoptosis or a mitotic inhibitor or anti-microtubule inhibitor, or an alkylating agent, or a topoisomerase inhibitor, or a glycopeptide antibiotic, or steroid receptor inhibitor, or a matrix metalloproteinase (MMP) inhibitor, or an mTOR (mammalian target of rapamycin) inhibitor, or a macrolide or a composition comprising a macrolide ring, an aromatase inhibitor,

and optionally the inducer of apoptosis or a mitotic inhibitor or anti-microtubule inhibitor comprises or consists of a raltitrexed or equivalent, or TOMUDEX™; a doxorubicin or equivalent, or ADRIAMYCIN™; a fluorouracil or 5-fluorouracil or equivalent; a paclitaxel or equivalent, or TAXOL™ or ABRAXANE™; a docetaxel or equivalent, or TAXOTERE™; a larotaxel, tesetaxel or ortataxel or equivalent; an epothilone or an epothilone A, B, C, D, E or F or equivalent; an ixabepilone (also known as azaepothilone B) or equivalent, or BMS-247550™; a vincristine (also known as leurocristine) or equivalent, or ONCOVIN™; a vinblastin, vinblastine, vindesine, vinflunine, vinorelbine or NAVELBINE™ or equivalent; or, any combination thereof,

and optionally the alkylating agent comprises or consists of a cisplatin or equivalent; a cisplatinum or equivalent; a cis-diamminedichloridoplatinum(II) (CDDP) or equivalent; a carboplatin or equivalent; a oxaloplatin or equivalent; a cyclophosphamide (cytophosphane) or equivalent, or ENDOXAN™, CYTOXAN™, NEOSAR™ or REVIMMUNE™; a mechlorethamine or equivalent; a chlormethine or equivalent; a mustine or equivalent; a nitrogen mustard or equivalent; a chlorambucil or equivalent, or LEUKERAN™; or, a combination thereof,

and optionally the topoisomerase inhibitor comprises or consists of an etoposide or equivalent, or EPOSIN™, ETOPOPHOS™, VEPESID™ or VP-16™; an amsacrine or equivalent; a topotecan or equivalent, or HYCAMTIN™; a teniposide or equivalent, or VUMON™ or VM-26™; an epipodophyllotoxin or equivalent; a camptothecin or equivalent; an irinotecan or equivalent, or CAMPTOSAR™; or, a combination thereof, and optionally the glycopeptide antibiotic comprises or consists of a bleomycin or equivalent or a bleomycin A₂ or B₂ or equivalent; a mitomycin or a mitomycin C or equivalent, a plicamycin (also known as mithramycin) or equivalent, or MITHRACIN™; or, a combination thereof,

and optionally the steroid receptor inhibitor comprises or consists of an estrogen receptor modulator (a SERM), and optionally the estrogen receptor modulator comprises or consists of a tamoxifen or equivalent, or NOLVADEX™, ISTUBAL™ or VALODEX™, and optionally the steroid inhibitor or an anti-steroid comprises or consists of a finasteride or equivalent, or PROSCAR™, PROPECIA™, FINCAR™, FINPECIA™ FINAX™ FINAST™, FINARA™ FINALO™ PROSTERIDE™, GEFINA™, APPECIA™, FINASTERID IVAX™, FINASTERID or ALTERNOVA™,

and optionally the macrolide or composition comprising a macrolide ring comprises or consists of a clarithromycin or equivalent, or BIAXIN™, KLARICID™, KLABAX™ CLARIPEN™ CLARIDAR™ FROMILID™ or CLACID™; an azithromycin or equivalent, or ZITHROMAX™, ZITROMAX™ or SUMAMED™; a dirithromycin or equivalent; an erythromycin or equivalent; a roxithromycin or equivalent, or ROXO™, SURLID™, RULIDE™, BIAXSIG™, ROXAR™, ROXIMYCIN™ or COROXIN™; a telithromycin or equivalent or KETEK™; a josamycin or equivalent; a kitasamycin or equivalent; a midecamycin or equivalent; oleandomycin or equivalent; a roxithromycin or equivalent, or ROXO™, SURLID™, RULIDE™, BIAXSIG™ ROXAR™ ROXIMYCIN™ or COROXIN™; a troleandomycin or equivalent; or a tylosin or equivalent; or, any combination thereof,

and optionally the aromatase inhibitor comprises: a 4-Hydroxyandrostenedione, a 1,4,6-Androstatrien-3,17-dione (ATD), or a 4-Androstene-3,6,17-trione (6-OXO);

(e) a radiotherapy enhancing agent;

(f) a combination of at least one beta adrenergic receptor antagonist and at least one non-steroidal anti-inflammatory drug (NSAID), or a propranolol and an etodolac, or VT-122™ (Vicus Therapeutics, Morristown, N.J.);

(g) an H₂-receptor antagonist (H₂RA),

wherein optionally the H₂-receptor antagonist comprises or consists of a cimetidine or equivalent, or TAGAMET™, TAGAMET HB™ or TAGAMET HB200™; a ranitidine or equivalent, or TRITEC™ or ZANTAC™; a famotidine or equivalent, or PEPCIDINE™ or PEPCID™; a nizatidine or equivalent, or TAZAC™ or AXID™;

(h) a proton pump inhibitor (a PPI), wherein optionally the proton pump inhibitor comprises or consists of a benzimidazole compound or structure, or an imidazopyridine compound or structure, and optionally the imidazopyridine compound or structure comprises or consists of a zolpidem or equivalent, or AMBIEN™, AMBIEN CR™, IVEDAL™, NYTAMEL™, STILNOCT™, STILNOX™, ZOLDEM™, ZOLNOD™ or ZOLPIHEXAL™; an alpidem (also called ananxyl) or equivalent; a saripidem or equivalent; necopidem or equivalent;

(i) a metformin, or an N,N-Dimethylimidodicarbonimidic diamide, or a GLUCOPHAGE™, FORTAMET™, GLUMETZA™ or RIOMET™, or a quinoline, an aminoquinoline, e.g., a 4-aminoquinoline or an 8-Aminoquinoline, e.g., a chloroquine (or ARALEN™), a hydroxychloroquine (or PLAQUENIL™) a quinacrine (ATABRINE™), a primaquine, a tafenoquine, or equivalents thereof; or

(j) any combination of (a) to (i).

In alternative embodiments, the anticancer agent or reagent comprises a radioactive particle or isotope; or a microscopic, radioactive glass microsphere; a plurality of radioactive glass microspheres, optionally about 20 to 30 micrometers in diameter; or, insoluble glass microspheres comprising a yttrium-90, or a THERASPHERE™ (Biocompatibles International, Surry UK). In alternative embodiments, the anticancer agent or reagent comprises a drug-eluting or a cancer drug-eluting particle, liposome or bead, or a doxorubicin-loaded drug-eluting bead, or a DC Bead®.

In alternative embodiments, the anticancer agent or reagent comprises: a sorafenib or equivalent, or NEXAVAR™; a sunitinib or equivalent, or SUTENT™; an erlotinib or equivalent, or TARCEVA™; an imatinib or equivalent, or GLEEVEC™; a lapatinib or equivalent, or TYKERB™; a toceranib or equivalent, or PALLADIA™; a masitinib or equivalent, or MASIVET™; a bevacizumab or equivalent, or AVASTIN™; a trastuzumab or equivalent, or HERCEPTIN™; a cetuximab or equivalent, or ERBITUX™; a bevacizumab or equivalent, or AVASTIN™ or BIBW 2992; a gefitinib or equivalent, or IRESSA™; a ranibizumab or equivalent, or LUCENTIS™; a pegaptanib or equivalent, or MACUGEN™; a dasatinib or equivalent, or BMS-354825™; a sunitinib or equivalent, or SUTENT™; a pazopanib or equivalent; a nilotinib or equivalent, or TASIGNA™; a panitumumab or equivalent, or VECTIBIX™; a bandetinib or equivalent; a brivanib or equivalent, or E7080™; a thalidomide or equivalent, or THALOMID™; lenalidomide or equivalent, or REVLIMID™; a bortezomib or equivalent, or VELCADE™; disulfiram or equivalent, or ANTABUSE™ or ANTABUS™; or an epigallocatechin gallate (EGCG) or equivalent; a demecolcine, an etoglucid or elsamitrucin, a lonidamine, a lucanthone, a mitotane or a mitoguazone or equivalent; or any combination thereof.

In alternative embodiments, the product of manufacture, device or composition comprises any combination of ingredients or agents, e.g., any combination of ingredients or agents as described herein.

In alternative embodiments, the product of manufacture, device or composition provided herein further comprises: (a) an adjuvant; (b) an immunostimulating cytokine or biologic; or (c) any combination of (a) or (b).

Provided are products of manufacture, devices or compositions provided herein in an in situ milieu or environment, e.g., in a tissue or an organ.

In alternative embodiments, provided are a device, a medical device, an implant, a breast implant, a prosthesis, a stent, a catheter, comprising a product of manufacture, device or composition provided herein.

In alternative embodiments, provided are methods for:

(a) (i) treating, preventing or ameliorating a tumor or a cancer,

(ii) vaccinating or immunizing an individual against an antigen or an immunogen,

(iii) vaccinating or immunizing an individual against a cancer or tumor antigen or immunogen, or a plurality of antigens or immunogens, or any combination thereof, (iv) immunostimulating an individual, or

(v) any combination of (i) to (iv),

comprising:

applying or administering to an individual in need thereof; or, applying or administering to a target cancer, tumor, tissue or organ, or an affected tissue or organ:
the product of manufacture, device or composition provided herein; or,
the device, medical device, implant, breast implant, prosthesis, stent or catheter provided herein;

In alternative embodiments, the method of (a) further comprises applying or administering to the individual in need thereof; or, applying or administering to the target cancer, tumor, tissue or organ, or affected tissue or organ, the product of manufacture, device or composition, or the device, medical device, implant, breast implant, prosthesis, stent or catheter, simultaneous with, in conjunction with, before and/or after a systemic therapy,

wherein optionally the product of manufacture, device or composition, or the device, medical device, implant, breast implant, prosthesis, stent or catheter is or are administered before the systemic therapy, or both are administered consecutively, or the product of manufacture, device or composition, or the device, medical device, implant, breast implant, prosthesis, stent or catheter is administered after the systemic therapy, or any combination thereof;

In alternative embodiments, the systemic therapy comprises:
(i) treating, preventing or ameliorating a tumor or a cancer,
(ii) vaccinating or immunizing an individual against an antigen or an immunogen,
(iii) vaccinating or immunizing an individual against a cancer or tumor antigen or immunogen, or a plurality of antigens or immunogens, or any combination thereof,
(iv) immunostimulating an individual,
(v) providing an anticancer or antitumor treatment, or
(vi) any combination of (i) to (v);

In alternative embodiments, the systemic therapy comprises a systemic anti-cancer or anti-tumor treatment, or an anti-cancer or anti-tumor immunotherapy or vaccination, or an anti-cancer or anti-tumor immunostimulation;

In alternative embodiments, the systemic anti-cancer or anti-tumor treatment comprises administration, application, or use of a drug, a biologic, a nutrient, an anti-cancer or anti-tumor dietary regimen, a radioactive agent, a tumor ablative agent, or a combination thereof;

In alternative embodiments, the systemic anti-cancer or anti-tumor treatment comprises administration, application, or use of an anti-cancer or anti-tumor radiotherapy or a proton beam therapy;

In alternative embodiments, wherein the systemic anti-cancer or anti-tumor treatment comprises administration, application, or use of a proton pump inhibitor (a PPI),

wherein optionally the proton pump inhibitor comprises or consists of a benzimidazole compound or structure, or an imidazopyridine compound or structure,

and optionally the imidazopyridine compound or structure comprises or consists of a zolpidem or equivalent, or AMBIEN™, AMBIEN CR™, IVEDAL™, NYTAMEL™, STILNOCT™, STILNOX™, ZOLDEM™, ZOLNOD™ or ZOLPIHEXAL™; an alpidem (also called ananxyl) or equivalent; a saripidem or equivalent; necopidem or equivalent;

In alternative embodiments, the systemic anti-cancer or anti-tumor treatment comprises administration, application, or use of an H₂-receptor antagonist (H₂RA),

wherein optionally the H₂-receptor antagonist comprises or consists of a cimetidine or equivalent, or TAGAMET™, TAGAMET HB™ or TAGAMET HB200™; a ranitidine or equivalent, or TRITEC™ or ZANTAC™; a famotidine or equivalent, or PEPCIDINE™ or PEPCID™; a nizatidine or equivalent, or TAZAC™ or AXID™;

In alternative embodiments, the systemic anti-cancer or anti-tumor treatment comprises administration, application, or use of a combination of at least one beta adrenergic receptor antagonist and at least one non-steroidal anti-inflammatory drug (NSAID), or a propranolol and an etodolac, or a VT-122™ (Vicus Therapeutics, Morristown, N.J.);

In alternative embodiments, the systemic anti-cancer or anti-tumor treatment comprises administration, application, or use of a cytokine,

wherein optionally the cytokine comprises an IL-2 or an interferon (IFN),

and optionally the interferon is an alpha-IFN or a gamma-IFN;

and optionally the IL-2 is a recombinant IL-2, an aldesleukin, or a PROLEUKIN (Prometheus Laboratories),

wherein optionally the IL-2, recombinant IL-2, or aldesleukin is dosages at about: 0.1 to 20, 1.0 to 20, 1 to 10, or 4 to 5, or 4.5 millions of IUs per cycle; or is dosaged for: 1 to 5, 2 to 4, or 3 cycles number of cycles of therapy;

In alternative embodiments, the systemic anti-cancer or anti-tumor treatment comprises administration, application, or use of a an immune checkpoint blockade agent, or an agent that blocks the interaction between a transmembrane programmed cell death 1 protein (PD-1; also known as CD279) and its ligand, PD-1 ligand 1 (PD-L1), or an ipilumumab (CTLA-4 mAb) or nivolumab (PD-1 mAb), or pembrolizumab (PD-1 mAb), or a lambrolizumab (a PD-L1 mAb);

In alternative embodiments, the systemic anti-cancer or anti-tumor treatment comprises administration, application, or use of an activator of a pattern recognition receptor (PRR) or a toll-like receptor 7 (TLR7), or an imiquimod;

In alternative embodiments, the systemic anti-cancer or anti-tumor treatment comprises administration, application, or use of a radiotherapy enhancing agent;

In alternative embodiments, the systemic anti-cancer or anti-tumor treatment comprises administration, application, or use of chemotherapeutic agent,

wherein optionally the chemotherapeutic agent comprises a doxorubicin or a carboplatin, or comprises an inducer of apoptosis or a mitotic inhibitor or anti-microtubule inhibitor, or an alkylating agent, or a topoisomerase inhibitor, or a glycopeptide antibiotic, or steroid receptor inhibitor, or a matrix metalloproteinase (MMP) inhibitor, or an mTOR (mammalian target of rapamycin) inhibitor, or a macrolide or a composition comprising a macrolide ring, an aromatase inhibitor;

In alternative embodiments, the systemic anti-cancer or anti-tumor treatment comprises administration, application, or use of:
(1) a systemic immunotherapy,
(2) a combination of at least one beta adrenergic receptor antagonist and at least one non-steroidal anti-inflammatory drug (NSAID), or a propranolol and an etodolac, or a VT-122™,
(3) a proton pump inhibitor (a PPI), and
(4) an H₂-receptor antagonist (H₂RA);

In alternative embodiments, the systemic anti-cancer or anti-tumor treatment comprises administration, application, or use of a chemotherapy and/or a radiotherapy, and use of a combination of at least one beta adrenergic receptor antagonist and at least one non-steroidal anti-inflammatory drug (NSAID), or a propranolol and an etodolac, or a VT-122™; or

In alternative embodiments, methods provided herein comprising any combination of therapies, treatments or drugs as described herein.

In alternative embodiments, provided are methods for treating, preventing or ameliorating a tumor or a cancer, comprising:

(a) applying or administering to an individual in need thereof; or, applying or administering to an effected tissue; the product of manufacture, device or composition provided herein; or, the device, medical device, implant, breast implant, prosthesis, stent or catheter provided herein; and

(b) administering to the individual in need thereof:
(i) a systemic anti-cancer or anti-tumor treatment,
wherein optionally the systemic anti-cancer or anti-tumor treatment comprises administration of a drug, a biologic, a cytokine, a nutrient, an anti-cancer or anti-tumor dietary regimen, a radioactive agent, a tumor ablative agent, or
(ii) an anti-cancer or anti-tumor radiotherapy or a proton beam therapy,

wherein the anti-cancer or anti-tumor treatment of (a) is administered before the anti-cancer or anti-tumor treatment of (b), or both are administered consecutively, or the anti-cancer or anti-tumor treatment of (a) is administered after the anti-cancer or anti-tumor treatment of (b), or any combination thereof.

In alternative embodiments, the cancer or tumor is: a mastocytoma or a mast cell tumor, an ovarian cancer, pancreatic cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer.

In alternative embodiments of the methods: the product of manufacture, device or composition provided herein; or, the device, medical device, implant, breast implant, prosthesis, stent or catheter provided herein, comprises:
(1) a combination of at least one beta adrenergic receptor antagonist and at least one non-steroidal anti-inflammatory drug (NSAID), or a propranolol and an etodolac, or a VT-122™ (Vicus Therapeutics, Morristown, N.J.),
wherein optionally the at least one beta adrenergic receptor antagonist and/or the at least one non-steroidal anti-inflammatory drug (NSAID) is/are administered locally or systemically, but separately from the product of manufacture, device or composition provided herein; or, the device, medical device, implant, breast implant, prosthesis, stent or catheter provided herein,
(2) a cytokine,
wherein optionally the cytokine comprises an IL-2 or an interferon (IFN),
and optionally the interferon is an alpha-IFN or a gamma-IFN;
and optionally the IL-2 is a recombinant IL-2, an aldesleukin, or a PROLEUKIN (Prometheus Laboratories),
wherein optionally the IL-2, recombinant IL-2, or aldesleukin is dosages at about: 0.1 to 20, 1.0 to 20, 1 to 10, or 4 to 5, or 4.5 millions of IUs per cycle; or is dosaged for: 1 to 5, 2 to 4, or 3 cycles number of cycles of therapy, or
wherein optionally the cytokine is administered locally, but separately from the product of manufacture, device or composition provided herein; or, the device, medical device, implant, breast implant, prosthesis, stent or catheter provided herein,
(3) a combination of (1) and (2).

In alternative embodiments of the methods, the systemic anti-cancer or anti-tumor treatment comprises an anti-cancer or anti-tumor radiotherapy or a proton beam therapy.

In alternative embodiments of the methods the at least one beta adrenergic receptor antagonist and at least one non-steroidal anti-inflammatory drug (NSAID), are administered systemically, and the cytokine, optionally IL-2, is administered with (or as part of) the product of manufacture, device or composition provided herein; or, the device, medical device, implant, breast implant, prosthesis, stent or catheter provided herein; or

(c) the method of (a) or (b), wherein the cancer being treated, prevented or ameliorated is a mast cell tumor or a melanoma.

In alternative embodiments, provided are kits, or integrated point of care mixing kits, comprising

(a) the product of manufacture, device or composition provided herein, or a sterile hydrogel material or sterile hydrogel provided herein, or as used in a product of manufacture or a device provided herein, or, the device, medical device, implant, breast implant, prosthesis, stent or catheter provided herein,

wherein optionally the sterile hydrogel material or sterile hydrogel is: (i) in a substantially liquid form capable of setting, gelling or self-assembling; (ii) a partially assembled or gelled hydrogel; or, (iii) in a set, gelled or self-assembled state; or a substantially set, gelled or self-assembled state;

(b) the of (a), kit further comprising instructions for practicing a method provided herein.

In alternative embodiments, provided are therapeutic combinations comprising: (a) (i) a product of manufacture, device, or composition provided herein (ii) a device, medical device, implant, breast implant, prosthesis, stent or catheter provided herein; (iii) a kit, or an integrated point of care mixing kit, provided herein; or, (iv) a plurality of compositions used to practice a method provided herein; and, (b) (i) a biologic, a drug or an immunostimulating agent or reagent, (ii) an antigen or an immunogen, or a plurality of antigens or immunogens, (iii) a biologic, a drug or an immunostimulating agent or reagent, and an antigen or an immunogen, or a plurality of antigens or immunogens, (iv) an anticancer agent or reagent, or (v) any combination thereof.

In alternative embodiments of the therapeutic combinations, the composition or compositions, or the biologics, drugs, immunostimulating agents or reagents, antigens or immunogens, or anticancer agents or reagents are systemically administered. In alternative embodiments, the composition or compositions, or the biologics, drugs, immunostimulating agents or reagents, antigens or immunogens, or anticancer agents or reagents comprise: a combination of at least one beta adrenergic receptor antagonist and at least one non-steroidal anti-inflammatory drug (NSAID), or a propranolol and an etodolac, or a VT-122™ (Vicus Therapeutics, Morristown, N.J.).

In alternative embodiments the therapeutic combinations provided herein are used in the treatment, amelioration or healing of: a cancer or a tumor. In alternative embodiments, the cancer or tumor is: a mastocytoma or a mast cell tumor, an ovarian cancer, pancreatic cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a Histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer.

In alternative embodiments, provided are uses of: (a) (i) a product of manufacture, device, or composition provided herein, (ii) a device, medical device, implant, breast implant, prosthesis, stent or catheter provided herein, (iii) a kit, or an integrated point of care mixing kit, provided herein; and/or (b) (i) a biologic, a drug or an immunostimulating agent or reagent, (ii) an antigen or an immunogen, or a plurality of antigens or immunogens, (iii) a biologic, a drug or an immunostimulating agent or reagent, and an antigen or an immunogen, or a plurality of antigens or immunogens, (iv) an anticancer or antitumor agent or reagent, (v) an anticancer or antitumor treatment, or (vi) any combination thereof, for: the treatment, amelioration, prevention or healing of: a cancer or a tumor. In alternative embodiments of the uses provided herein, the composition or compositions, or the biologics, drugs, immunostimulating agents or reagents, antigens or immunogens, or anticancer agents or reagents, of step (b), are systemically administered. In alternative embodiments, the composition or compositions, or the biologics, drugs, immunostimulating agents or reagents, antigens or immunogens, or anticancer agents or reagents, of step (b), comprises: a combination of at least one beta adrenergic receptor antagonist and at least one non-steroidal anti-inflammatory drug (NSAID), or a propranolol and an etodolac, or a VT-122™ (Vicus Therapeutics, Morristown, N.J.).

In alternative embodiments of the uses provided herein, the cancer or tumor is: a mastocytoma or a mast cell tumor, an ovarian cancer, pancreatic cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a Histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer.

In alternative embodiments of the uses provided herein, the systemic anti-cancer or anti-tumor treatment comprises administration, application, or use of a chemotherapy and/or a radiotherapy, and use of a combination of at least one beta adrenergic receptor antagonist and at least one non-steroidal anti-inflammatory drug (NSAID), or a propranolol and an etodolac, or a VT-122™.

In alternative embodiments of the uses provided herein, the systemic anti-cancer or anti-tumor treatment comprises administration, application, or use of: (1) a systemic immunotherapy, (2) a combination of at least one beta adrenergic receptor antagonist and at least one non-steroidal anti-inflammatory drug (NSAID), or a propranolol and an etodolac, or a VT-122™, (3) a proton pump inhibitor (a PPI), and (4) an H₂-receptor antagonist (H₂RA).

In alternative embodiments of the uses provided herein: the product of manufacture, device or composition provided herein; or, the device, medical device, implant, breast implant, prosthesis, stent or catheter provided herein, comprises: (1) a combination of at least one beta adrenergic receptor antagonist and at least one non-steroidal anti-inflammatory drug (NSAID), or a propranolol and an etodolac, or a VT-122™ (Vicus Therapeutics, Morristown, N.J.), wherein optionally the at least one beta adrenergic receptor antagonist and at least one non-steroidal anti-inflammatory drug (NSAID) is administered locally, but separately from the product of manufacture, device or composition provided herein; or, the device, medical device, implant, breast implant, prosthesis, stent or catheter provided herein, (2) a cytokine, wherein optionally the cytokine comprises an IL-2 or an interferon (IFN), and optionally the interferon is an alpha-IFN or a gamma-IFN; and optionally the IL-2 is a recombinant IL-2, an aldesleukin, or a PROLEUKIN (Prometheus Laboratories), wherein optionally the IL-2, recombinant IL-2, or aldesleukin is dosages at about: 0.1 to 20, 1.0 to 20, 1 to 10, or 4 to 5, or 4.5 millions of IUs per cycle; or is dosaged for: 1 to 5, 2 to 4, or 3 cycles number of cycles of therapy, or, (3) a combination of (1) and (2), wherein optionally the cytokine is administered locally, but separately from the product of manufacture, device or composition provided herein; or, the device, medical device, implant, breast implant, prosthesis, stent or catheter provided herein.

In alternative embodiments of the uses provided herein, the systemic anti-cancer or anti-tumor treatment comprises an anti-cancer or anti-tumor radiotherapy or a proton beam therapy; the at least one beta adrenergic receptor antagonist and at least one non-steroidal anti-inflammatory drug (NSAID), are administered systemically, and the cytokine, optionally IL-2, is administered with (or as part of) the product of manufacture, device or composition provided herein; or, the device, medical device, implant, breast implant, prosthesis, stent or catheter provided herein; and optionally, the cancer being treated, prevented or ameliorated is a mast cell tumor or a melanoma.

The details of one or more aspects of the invention are set forth in the description below. Other features, objects, and advantages of the invention will be apparent from the description and from the claims.

All publications, patents and patent applications cited herein are hereby expressly incorporated by reference for all purposes.

### BRIEF DESCRIPTION OF THE DRAWINGS

FIG. 1A (FIG. 1A) graphically illustrates Serum IL-2 in Mice Dosed with IL-2 in Water (with different mice designated numbers 151 through 155), as described in detail in Example 1, below.

FIG. 1B (FIG. 1B) graphically illustrates Serum IL-2 in Mice Dosed with IL-2 in 0.5% Purastat (with different mice designated numbers 251 through 254), as described in detail in Example 1, below.

FIG. 1C (FIG. 1C) graphically illustrates Serum IL-2 in Mice Dosed with IL-2 in 1.5% Purastat (with different mice designated numbers 351 through 355), as described in detail in Example 1, below.

FIG. 1D (FIG. 1D) graphically illustrates a summary of the data of FIGS. 1A, 1B and 1C, where the data demonstrates that Mice Dosed with IL-2 in 1.5% Purastat have higher sustained serum levels of IL-2 for a longer period of time, as described in detail in Example 1, below.

FIGS. 1E and 1F (FIG. 1E and FIG. 1F) summarize data from these studies, as illustrated in FIGS. 1A to 1D, as described in detail in Example 1, below.

FIG. 2 illustrates Table 1, the study design of Example 2, as described in detail in Example 2, below.

FIG. 3 graphically illustrates mean body weight on different study days for Group 1, Group 2 and Group 3, as described in detail in Example 2, below.

FIG. 4 illustrates Table 2, Example 2. Body Weight, as described in detail in Example 2, below.

FIG. 5 graphically illustrates survival curves of Group 1, Group 2 and Group 3, as described in detail in Example 2, below.

FIG. 6 illustrates Table 3, Example 2, Clinical Observations, as described in detail in Example 2, below.

FIG. 7 illustrates Table 4, Example 2, Tumor Measurements, as described in detail in Example 2, below.

FIG. 8 graphically illustrates tumor sizes on study days in Group 1, Group 2 and Group 3, as described in detail in Example 2, below.

FIG. 9 graphically illustrates a summary of tumor sizes as observed prior to tumor removal (Day 7), in each Group 1, Group 2 and Group 3, as described in detail in Example 2, below.

FIG. 10 illustrates Table 5, Example 2. Necropsy Observations, as described in detail in Example 2, below.

FIG. 11 illustrates Table 6, Example 2, the Histology Summary by Group, as described in detail in Example 2, below.

Reference will now be made in detail to various exemplary embodiments of the invention. The following detailed description is provided to give the reader a better understanding of certain details of aspects and embodiments of the invention, and should not be interpreted as a limitation on the scope of the invention.

### DETAILED DESCRIPTION

In alternative embodiments, provided are pharmaceutical compositions, formulations, kits and other products of manufacture, comprising a sterile hydrogel comprising a hydrogel material and one or a plurality of compositions or compounds, which may comprise: a biologic, a drug or an immunostimulating agent or reagent; an antigen or an immunogen, or a plurality of antigens or immunogens; an anticancer agent or reagent, or any combination thereof.

In alternative embodiments, the hydrogel or hydrogel material comprises a self-assembling peptide, e.g., a plurality of synthetic peptides characterized by stable B-sheet structure with ionic side-chain interactions after setting, gelling or self-assembling. In alternative embodiments, the hydrogel or hydrogel material comprises a 16-amino acid synthetic peptide (Ac-[RADA]₄-CONH₂), or SEQ ID NO:1, and optionally the hydrogel comprises PURAMATRIX™ (PuraMatrix™) (BD Biosciences, San Jose, Calif.) (or PURASTAT™ (PuraStat™) (3D Matrix Group, Tokyo, Japan)), or PURADERM™ (PuraDerm™) (3DMatrix, Ltd, Tokyo, Japan).

In alternative embodiments, the hydrogel comprises, or is mixed with: immunostimulating products, such as cytokines, toll-like receptors, immune check point inhibitors; tissue vaccines such as cancer immunogens; or, a combination of tissue vaccines and immunostimulating products. In alternative embodiments, a hydrogel-comprising product of manufacture, device or composition as provided herein is administered (e.g., administered locally, e.g., into, approximate to, or near, a tumor or lesion site) in conjunction with a systemic treatment, e.g., administered before, during and/or after the systemic treatment. In alternative embodiments, the systemic treatment (used in conjunction with a hydrogel-comprising product of manufacture, device or composition provided herein) comprises a systemic anti-cancer or anti-tumor treatment, e.g., comprising administration, application, or use of a chemotherapy, a radiation therapy, an radiosensitizing therapy, an ablation or surgical therapy, an immunotherapy, a diet or nutritional therapy, and the like. For example, in alternative embodiments, the systemic treatment (used in conjunction with a hydrogel-comprising product of manufacture, device or composition provided herein) comprises a combination of at least one beta adrenergic receptor antagonist and at least one non-steroidal anti-inflammatory drug (NSAID), or a propranolol and an etodolac, or a VT-122™ (Vicus Therapeutics, Morristown, N.J.). In another exemplary alternative embodiment, the hydrogel-comprising product of manufacture, device or composition provided herein comprise(s) an IL-2, such as a recombinant IL-2, an aldesleukin, or a PROLEUKIN (Prometheus Laboratories) (e.g., 10 mg/kg human IL-2 in 2% PURASTAT™ (PuraStat™) (BD Biosciences, San Jose, Calif.) (or PURAMATRIX™ (PuraMatrix™)), and the systemic treatment comprises administration, application, or use of: (1) a systemic immunotherapy, (2) a combination of at least one beta adrenergic receptor antagonist and at least one non-steroidal anti-inflammatory drug (NSAID), or a propranolol and an etodolac, or a VT-122™; and optionally also a proton pump inhibitor (a PPI), and/or an H₂-receptor antagonist (H₂RA).

### Hydrogel and Hydrogel Materials

In alternative embodiments, the hydrogel or hydrogel material comprises a self-assembling peptide. In alternative embodiments, the hydrogel or hydrogel material comprises a plurality of synthetic peptides characterized by stable B-sheet structure with ionic side-chain interactions after setting, gelling or self-assembling. In alternative embodiments, the hydrogel or hydrogel material comprises a self-assembling peptide comprising: the sequence Ile-Glu-Ile-Lys-Ile-Glu-Ile-Lys-Ile-Glu-Ile-Lys-Ile (IEIK)₃I (SEQ ID NO:3); or, the sequence Lys-Leu-Asp-Leu-Lys-Leu-Asp-Leu-Lys-Leu-Asp-Leu (KLDL)₃ (SEQ ID NO:2); or, a 16-amino acid synthetic peptide (Ac-[RADA]₄-CONH₂), or SEQ ID NO:1, which optionally can be or comprise a PURAMATRIX™ (PuraMatrix™) (BD Biosciences, San Jose, Calif.), a PURASTAT™ (PuraStat™) (BD Biosciences, San Jose, Calif.), or a PURADERM™ (PuraDerm™) (3DMatrix, Ltd, Tokyo, Japan), or equivalents.

PURAMATRIX™ (PuraMatrix™) and PURASTAT™ (PuraStat™) comprise a laboratory-designed, 16-amino acid polypeptide with a repeating sequence of arginine, alanine, and aspartic acid, or RADARADARADARADA (termed RADA₄ or [RADA]₄) (SEQ ID NO:1). The alternating positively and negatively charged amino acids (arginine and aspartic acid), along with the non-polar alanines in-between the charged amino acids, create two distinct structural surfaces, one hydrophilic and the other hydrophobic (Zhang and Altman, 1999[5]). The RADA polypeptide monomer building blocks form β-sheet structures upon exposure to physiological concentrations of salt, i.e., tissue culture media or physiological fluids such as blood, via complementary ionic bond formation at the hydrophilic surface of the molecules (Hauser, et al. 2010 [3]).

With regard to fibril formation, the hydrophobic sides of the peptide form a double sheet inside of the fibers and the hydrophilic side forms the outside of the nanofibers that interact with water molecules, forming an extremely high water content hydrogel; for example, in one embodiment, a PURASTAT® (PuraStat®) or equivalent hydrogel comprising 2.5% RADA peptide or equivalent and 97.5% water is used to practice the invention.

PURASTAT® (PuraStat®), based on the self-assembling peptide platform technology of PURAMATRIX™ (PuraMatrix™), is a CE (Conformity Europeenne, meaning “European Conformity”) mark approved surgical hemostatic agent. PuraStat® is safe, synthetic, non-biogenic, biocompatible, resorbable peptide hydrogel with no risk of transmissible spongiform encephalopathy (TSE) transmission. PURASTAT® (PuraStat®), a fully transparent slightly viscous aqueous peptide (2.5%) solution, is sold in a pre-filled syringe and is currently available in 1 mL, 3 mL and 5 mL unit doses indicated for hemostasis in several surgical circumstances.

Provided are processes of making a hydrogel comprising a biologic, a drug or an immunostimulating agent or reagent, an antigen or an immunogen, or a plurality of antigens or immunogens, an anticancer agent or reagent, or any combination thereof, mixed with exemplary self-assembly hydrogels, e.g. self-assembling peptide hydrogels such as a PURAMATRIX™ (PuraMatrix™) (BD Biosciences, San Jose, Calif.), or a PURADERM™ (PuraDerm™) (3DMatrix, Ltd, Tokyo, Japan).

In alternative embodiments, antibiotics or other drugs are also used (e.g., are mixed) with a biologic, a drug or an immunostimulating agent or reagent, an antigen or an immunogen, or a plurality of antigens or immunogens, an anticancer agent or reagent, or any combination thereof, in the hydrogel. Any antibiotic and/or any other biologic, drug or immunostimulating agent or reagent, antigen or immunogen, or anticancer agent or reagent, can be included in the hydrogel.

### Harvesting of Tumor Antigens

In alternative embodiments, pharmaceutical compositions, formulations, kits and other products of manufacture, comprise a sterile hydrogel comprising a hydrogel material and a cancer or tumor antigen, immunogen, or a plurality of antigens or immunogens, or any combination thereof, which can be a cancer or a tumor cell extract, or a processed cancer or tumor cell. In alternative embodiments, the processed cancer or tumor cell is a minced cancer or tumor tissue or cell, and optionally the cancer or tumor tissue is minced with a device for making a mixed thickness skin micrograft or a split-thickness skin graft, or an XPANSION® device or an XPANSION MICROGRAFTING SYSTEM® (SteadMed Medical, Fort Worth, Tex.).

The present invention is further defined in the following Examples. It should be understood that these examples, while indicating preferred embodiments of the invention, are given by way of illustration only and are not to be construed as limiting in any manner. From the above discussion and these examples, one skilled in the art can ascertain the essential characteristics of this invention, and without departing from the spirit and scope thereof, can make various changes and modifications of the invention to adapt it to various usages and conditions.

### Examples

### Example 1: Exemplary Hydrogel Compositions and Methods for Making them

The following example describes an exemplary product of manufacture/device as provided herein comprising an IL-2. The objective of this study is to assess the pharmacokinetics of IL-2 following subcutaneous injection of an exemplary IL-2-comprising hydrogel as provided herein in mice.

### Test and Articles:

The test article is an exemplary product of manufacture/device as provided herein comprising IL-2; in particular, PURASTAT™ (PuraStat™) (BD Biosciences, San Jose, Calif.) hydrogel and recombinant IL-2 (or rhIL-2, i.e., aldesleukin) This is formulated prior to dosing. This test article is provided at two concentrations: 5 μg/mL and 50 μg/mL of PURASTAT™ hydrogel.

### Test System:

The study is performed using a total of 10 male or female C57BL/6 mice (Mus musculus) (approximately 8-10 weeks of age, 16-20 g each, at the time of dosing), obtained from Simonsen Laboratories (Gilroy, Calif.), Charles River (Wilmington, Mass.), or other approved vendor. Animals are acclimated for at least three days before dose administration. The animals are group-housed (at up to 5 per cage) in plastic “shoe-box” mouse cages in a room dedicated to rodents. LabDiet® 5001 Rodent Diet (Purina Mills, Inc., St. Louis, Mo.) or other approved diet is provided ad libitum throughout the acclimation and treatment phases. Fresh tap water from the Sunnyvale Municipal Water Supply is provided ad libitum to the animals via water bottles.

Twelve hours of light and twelve hours of dark is provided in the animal rooms.

### Study Design:

The study design is summarized in Table 1.

Prior to dosing, the mice are weighed and assigned to two groups of 5 each. On Day 0, animals of Group 1, 2 and 3 are dosed by subcutaneous (SC) injection of 10 μg of IL-2 in 0.2 mL (50 μg IL-2/mL) of Sterile Water, 0.5% or 1.5% of Purastat Hydrogel 1.5%. Blood for serum is collected prior to dosing (“−24 hr”) and at 1, 2, 4 and 8 hours after dose administration. Blood is collected via the facial vein, except for the final (8-hour) bleed, which is performed via terminal cardiocentesis. Sufficient blood is collected from each animal at each time point to yield a minimum of 25 μL of serum per sample. Serum samples are diluted 1:1 with PBS. Diluted serum specimens are kept frozen at −80® C. pending shipment to the analytical laboratory (Eve Technologies Corporation, Calgary, Alberta, Canada) for cytokines bioanalysis (Human Primary Cytokine Array/Chemokine Array 41-Plex Panel (EGF, Eotaxin-1, FGF-2, Flt-3L, Fractalkine, G-CSF, GM-CSF, GRO(pan), IFNα2, IFNγ, IL-1α, IL-10, IL-1ra, IL-2, IL-3, IL-4, IL-5, IL-6, IL-7, IL-8, IL-9, IL-10, IL-12 (p40), IL-12 (p70), IL-13, IL-15, IL-17A, IP-10, MCP-1, MCP-3, MDC, MIP-1α, MIP-1β, PDGF-AA, PDGF-AB/BB, RANTES, sCD40L, TGFα, TNFα, TNFβ, VEGF-A).

Following terminal blood collection mice are euthanized, and discarded. Necropsies are not planned, except for any animals that are found dead or moribund sacrificed during the study.

TABLE 1
Study Design

| | | | | | Bleeds |
|---|---|---|---|---|---|
| | Animal No. | In-life Treatment | | | (>25 μL serum |
| Group | (males) | Test Article | IL-2 | Route | per sample) |
| 1 | 101-105 | 0.2 mL | 10 μg | SC | Pre-dose and at 1, |
| | | Sterile Water | | | 2, 4 and 8 hours |
| 2 | 201-205 | 0.2 mL | | | |
| | | Purastat 0.5% | | | |
| 3 | 3-1-305 | 0.2 mL | | | |
| | | Purastat 1.5% | | | |

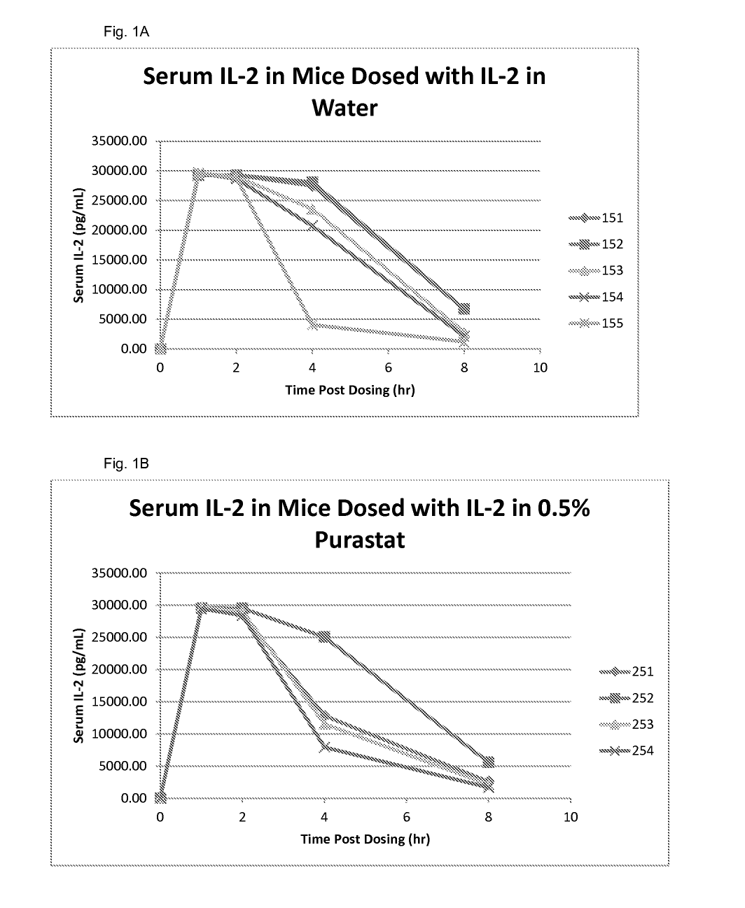

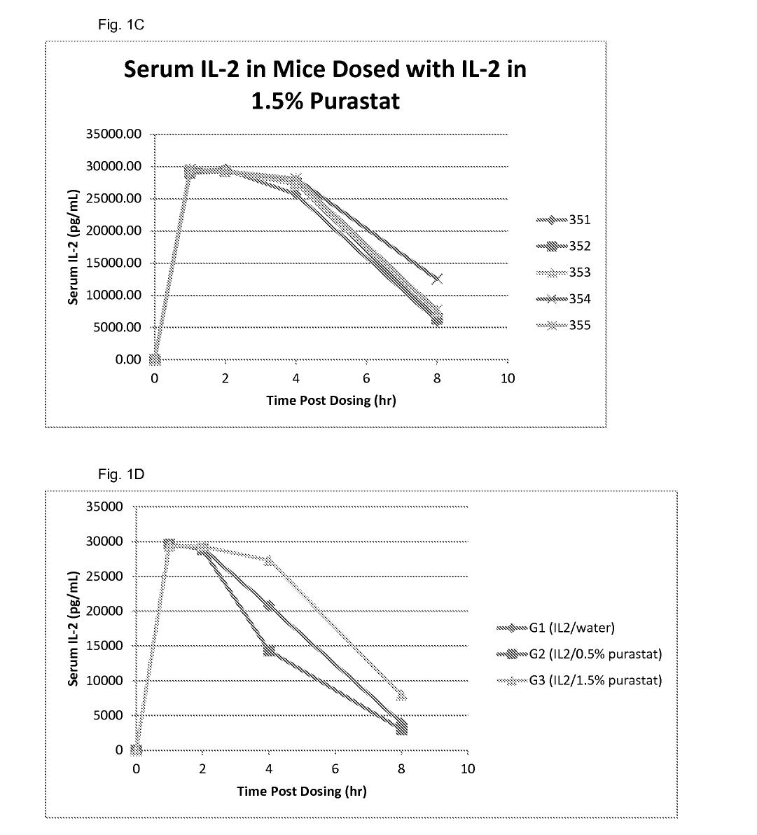

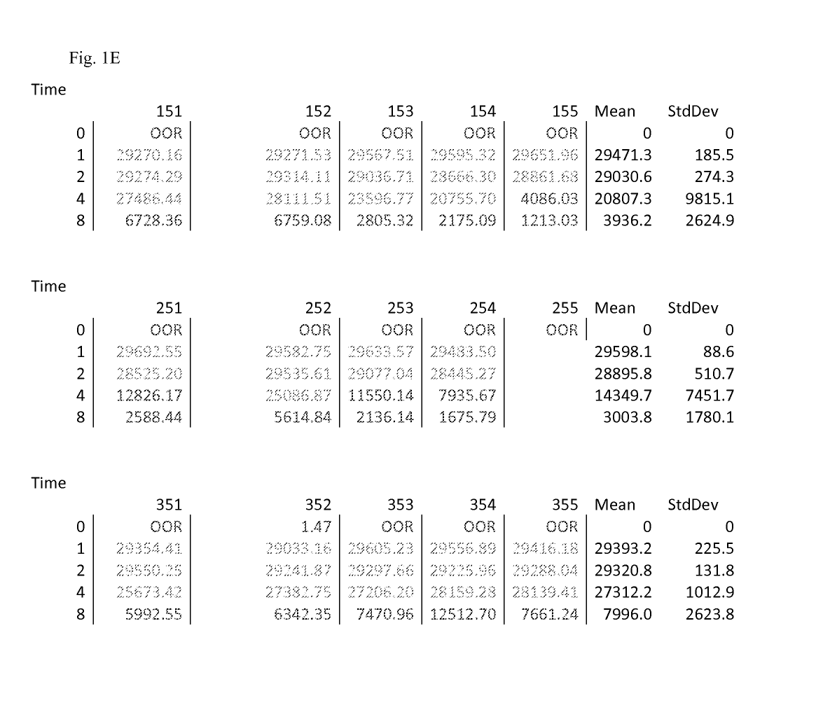

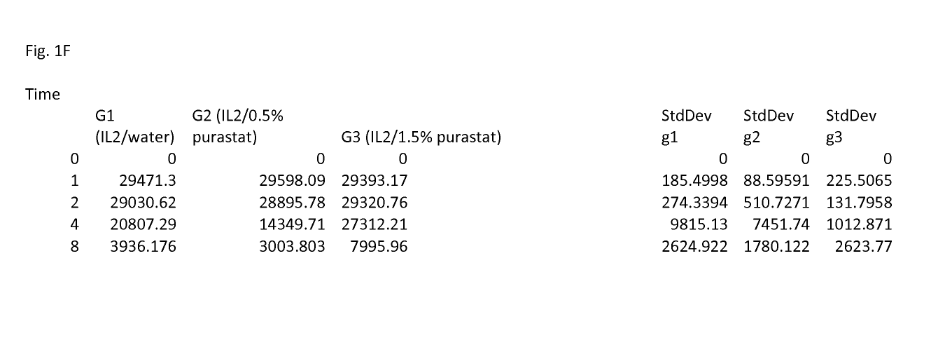

FIG. 1A (FIG. 1A) graphically illustrates Serum IL-2 in Mice Dosed with IL-2 in Water (with different mice designated numbers 151 through 155).

FIG. 1B (FIG. 1B) graphically illustrates Serum IL-2 in Mice Dosed with IL-2 in 0.5% Purastat (with different mice designated numbers 251 through 254).

FIG. 1C (FIG. 1C) graphically illustrates Serum IL-2 in Mice Dosed with IL-2 in 1.5% Purastat (with different mice designated numbers 351 through 355).

FIG. 1D (FIG. 1D) graphically illustrates a summary of the data of FIGS. 1A, 1B and 1C, where the data demonstrates that Mice Dosed with IL-2 in 1.5% Purastat have higher sustained serum levels of IL-2 for a longer period of time.

FIGS. 1E and 1F (FIG. 1E and FIG. 1F) summarize data from these studies, as illustrated in FIGS. 1A to 1D.

### Example 2: Exemplary Hydrogel Compositions and Methods for Using them

The following example describes studies with data demonstrating the efficacy of an exemplary product of manufacture/device as provided herein comprising an IL-2. This study used the art accepted B16-F1 Mouse Melanoma Model.

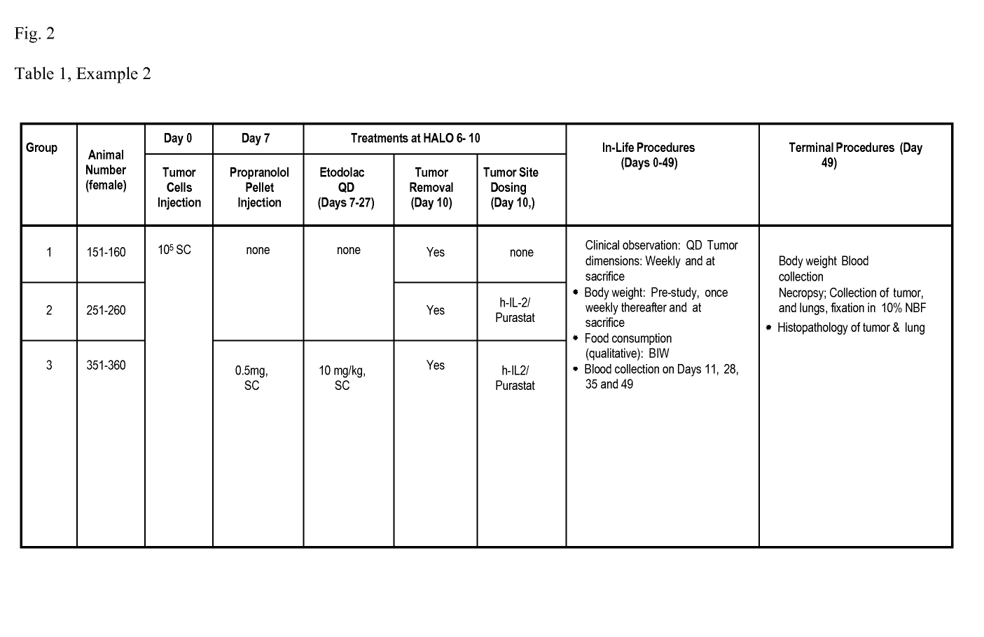

The study design is summarized in Table 1, FIG. 2.

### Material and Methods

The etodolac (a nonsteroidal anti-inflammatory drug) used: Taro Pharmaceuticals U.S.A., Inc. Haifa, Israel, 300 mg capsules. The etodolac was stored at controlled room temperature.

Propranolol (a sympatholytic non-selective beta blocker) used: a 21 day release pellet of 0.5 mg/pellet (Innovative Research of America, Catalog No. C-361, exp. 5/2017). Propranolol pellets were stored refrigerated (2-8® C.) pending use.

The PURASTAT™ hydrogel (a hydrogel material comprising a 16-amino acid synthetic peptide (Ac-[RADA]₄-CONH₂) (SEQ ID NO:1)) used: five 1-mL syringes of 2.5% PURASTAT™ catalog EM416 (Lot 13C08A10) (3D Matrix Group, Tokyo, Japan). PURASTAT™ syringes were stored refrigerated (2® C. to 8® C.) pending use.

The control article was corn oil, which was used as a vehicle for the first test article suspension; was obtained from Sigma Life Sciences (St. Louis, Mo.) as Catalog C8267-500ML, Lot MKBL8756V; control article was stored at controlled room temperature.

### Dose Preparation:

Etodolac:

A suspension of 5 mg/mL dosing solution of Etodolac was prepared by mixing the continent of a capsule (300 mg) in corn oil (60 mL).

PURASTAT™/IL-2 Mixture:

250 μg/mL of IL-2 was prepared in 100 mM acetic acid. 50 μg/ml of IL-2 in 2% PURASTAT™ was prepared as followed: 0.8 mL of 2.5% PURASTAT™ was liquefy by passing through 30 gauge needle and injected into 3 mL luer lock syringe and 0.2 mL of 250 μg/mL IL-2 was loaded into 1 mL syringe. Air bubbles were removed from each syringe and both syringes were connected by female-to-female luer lock connector. The two solutions were mixed by pushing the PURASTAT™ and the IL-2 back and forth for 6 times until fully mixed. 50 μg/ml solution of IL-2 in 0.2% PURASTAT™ prepared by mixing 0.2 mL of PURASTAT™ and 0.8 mL of 62.5 μg/mL of IL-2 in a similar way as described above.

### Test System:

Thirty four (34) females C57BL/6N mice (Mus musculus, 18-21 g each, at the time of arrival), were received from Simonsen Laboratories (Gilroy, Calif.) on 16 May 2014 and acclimated for eleven days prior to entry onto the study. During the acclimation period, the animals were observed at least once daily for clinical signs of abnormality. The animals were group-housed (at up to 5 per cage) in plastic “shoe-box” mouse cages in a room dedicated to rodents.

Light Cycle: Twelve hours of light and twelve hours of dark were provided in the animal rooms. A fluorescent light source was used, with lights turned on at approximately 5:00 AM and turned off at approximately 05:00 PM each day.

Feed and Water: LABDIET® 5001 Rodent Diet (Purina Mills, Inc., St. Louis, Mo.) or other approved diet was provided ad libitum throughout the acclimation and treatment phases. Fresh tap water from the Sunnyvale Municipal Water Supply will be provided ad libitum to the animals via water bottles.

### Study Design:

The study design is summarized in Table 1, FIG. 2. The study consisted of three groups of ten female C57BL/6 mice each. On Day 0, between 6 to 10 hours after light onset (HALO 6-10), mice were implanted with B16-F1 cells, a metastatic mouse melanoma cell line. Each mouse was implanted (subcutaneously (SC) on the cephalad dorsum with 1×10⁵ cells (0.1 mL) of the melanoma cells suspended in 50% Matrigel (BD Biosciences, Bedford, Mass.) in phosphate-buffered saline (PBS). Animals were returned to their cages and tumors allowed to develop for 7 days. On Day 7, thirty (30) mice of moderate body weight and harboring tumors of the desired size were allocated to three groups of 10 mice each.

Starting on Day 7 at the light intensity HALO 6-10 and continuing for a total of twenty one consecutive days, mice of Group 3 will be dosed once daily (QD) by SC injection of 10 mg/kg (˜0.2 mg/mouse) etodolac (Eto) and single injection of a 0.5 mg propranolol pellet.

On Day 10, when tumor diameter had reached 3-5 mm in diameter, at HALO 6-10, the primary B16-F1 tumor was surgically removed. Under isoflurane anesthesia, animals of Groups 1-3 were subjected to aseptic tumor removal surgery using an electrocautery (leaving about 1 mm³ of the original tumor).

For animals of Groups 2 and 3 only, 10 mg/kg human IL-2 in 2% PURASTAT™ hydrogel applied immediately after the removal of the tumor to the dissected area. The incision was closed with steel staples. The animals were recovered and observed daily; appropriate post-surgical care provided.

Blood for serum was collected one day after surgery (Day 11), again on Days 28, 35, prior to morbid sacrifice and at necropsy; blood was collected via the facial vein, except for the final bleed, which was performed via terminal cardiocentesis. Resulted serum specimens kept frozen at −80® C. pending shipment to the analytical laboratory (Eve Technologies Corporation, Calgary, Alberta, Canada) for mouse cytokines bioanalysis.

During the in-life period clinical observations were recorded at least once daily, body weight and tumor size (dimensions) was measured once weekly and at necropsy; qualitative food consumption was measured twice weekly (BIW).

On Day 49 the mice are euthanized. At necropsy, animals were weighed; the tumor and the surrounding tissue and lungs will be harvested and weighed. The tumor and the lungs were fixed in 10% neutral buffered formalin (NBF) for histological processing and examination. NBF fixed tissues were evaluated microscopically by a board-certified veterinary pathologist. Remaining tissues were discarded without further examination.

### Results:

Acclimation: There were no clinical signs of abnormality during the acclimation period. All animals were released for use in the study at the end of the acclimation period.

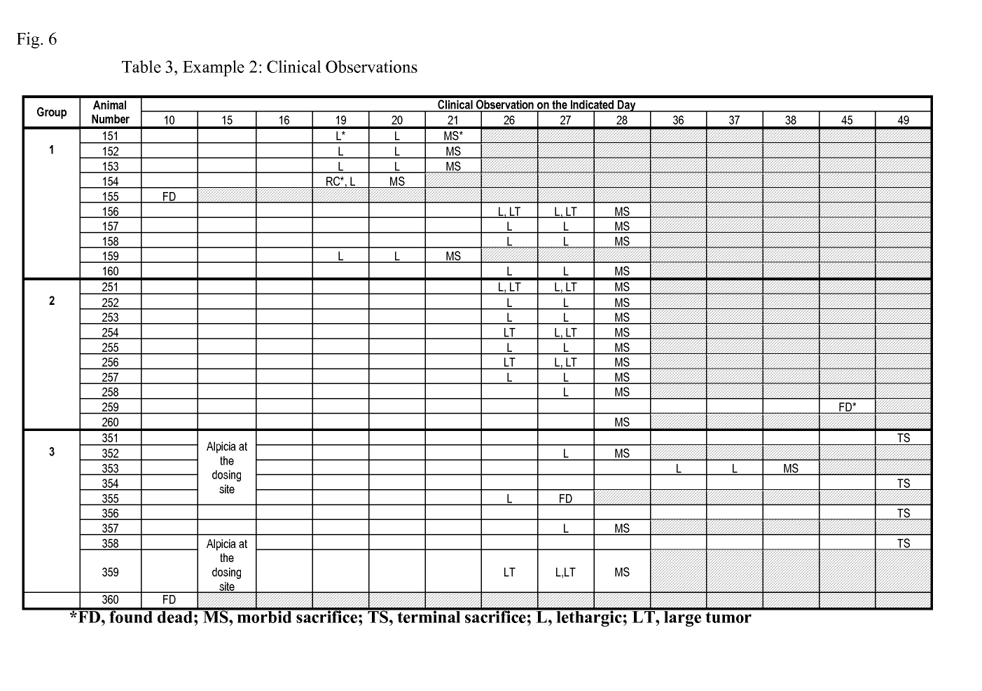

Clinical Observations: Clinical observations were recorded daily and are presented in Table 3 (FIG. 6). Starting on Day 19 (9 days after surgery), clinical signs such as lethargy and rough coat were observed. During the course of the study, twenty one mice were found to be moribund or having a large tumor. Per IACUC protocol and after veterinary consultation these animals were euthanized according to Testing Facility SOPs.

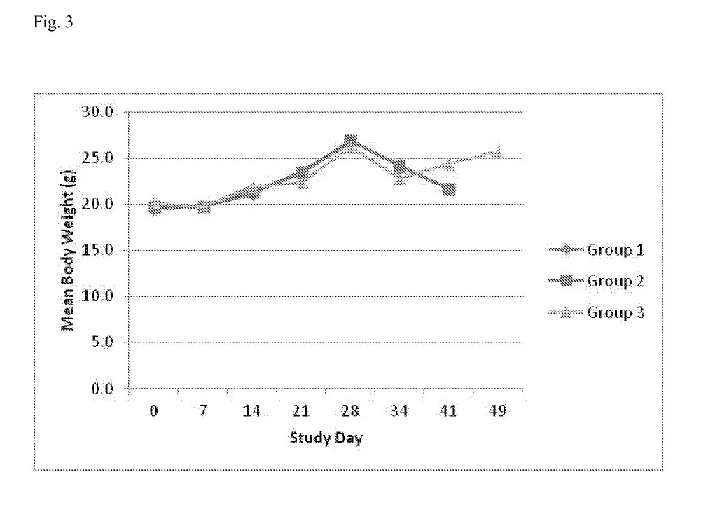

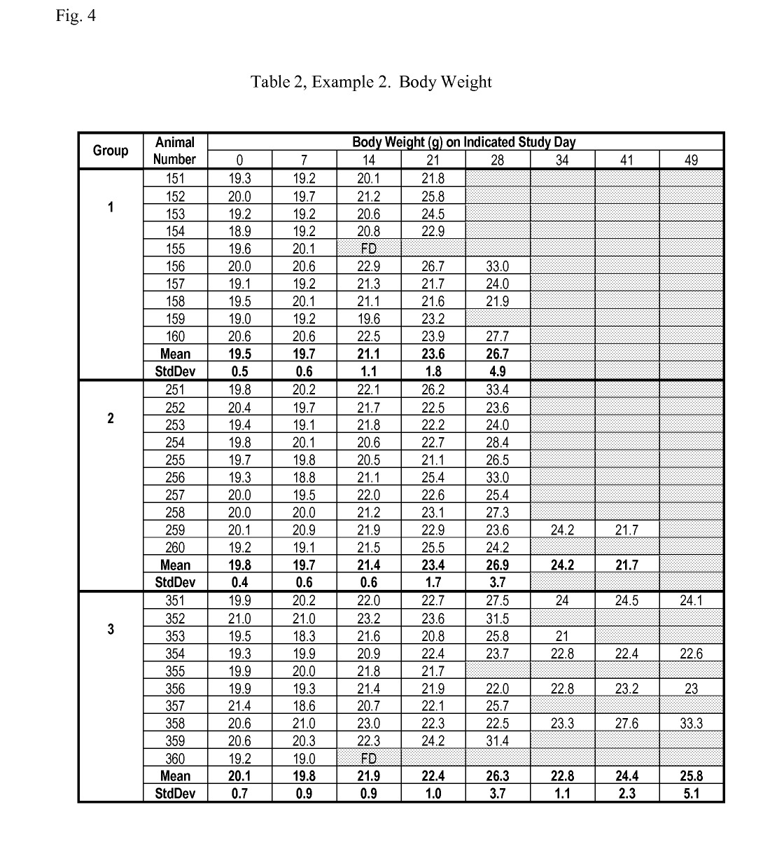

Body Weight: The mice were weighed prior to tumor implantation (Day 0), once weekly thereafter, and at sacrifice. Body weights are presented in Table 2, FIG. 4, and plotted in the graphic illustrated in FIG. 3. All animals gained weight during the course of the study; weight gains were similar in all groups and may be the result of tumor over growth. Starting on Day 28, a decrease in body weight was observed in the survived mice; these mice have small or no tumor which is probably the explanation to a decrease in body weight.

Mortality and Survival Curve: Two mice (Animals No. 155 and 360) died on Day 10, immediately after tumor removal surgery. No significant observations were found during the post-mortem examination of these mice.

Following tumor removal, twenty one mice were sacrificed for humane reasons due to excessively large tumors (see Table 3, FIG. 6).

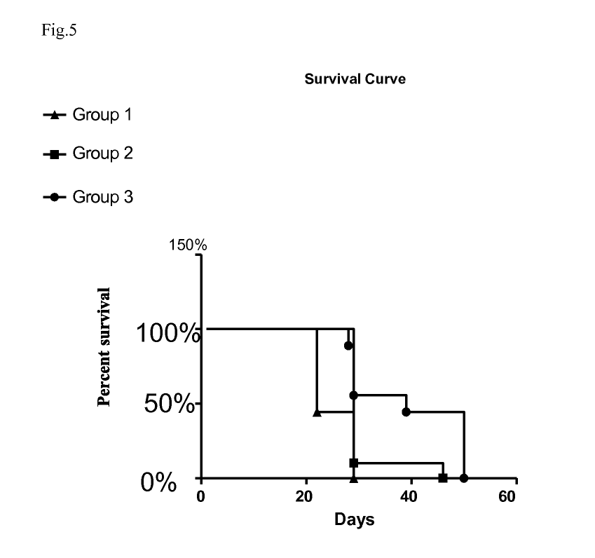

Kaplan-Meier survival curve was generated for individual groups and plotted in FIG. 5. Statistical analysis using a log rank (Mantel-Cox) test did not reveal significant differences in survival between Groups 1 and 2, but did show a significant difference in survival rate when compared Group 3 to Group 1.

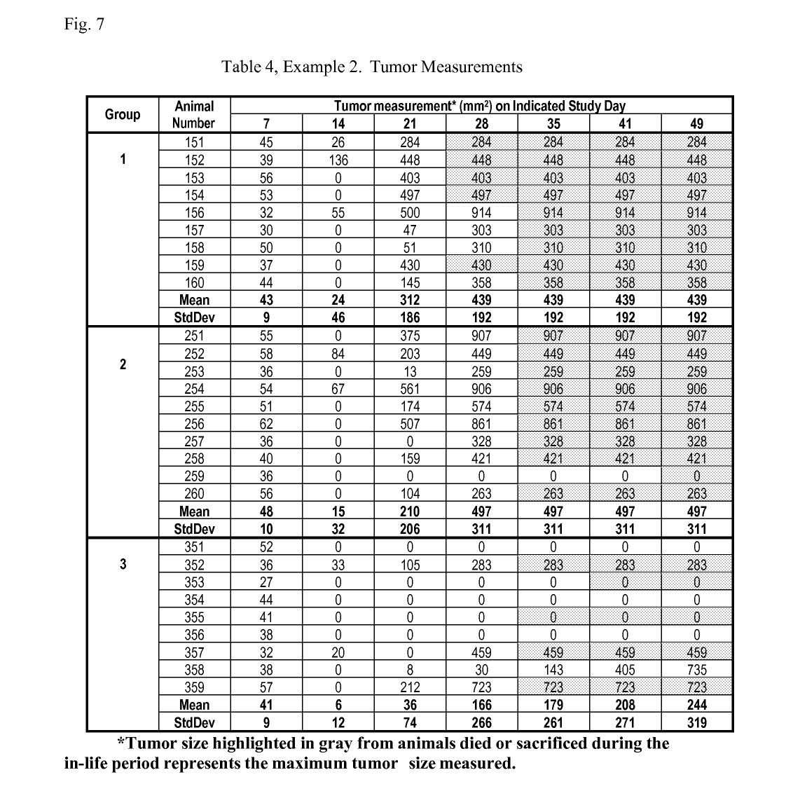

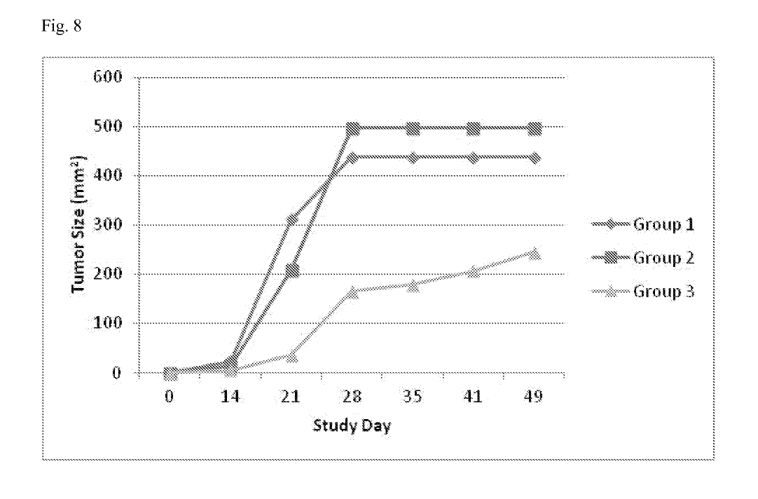

Tumor Size: During the course of the study, tumor dimensions were measured once weekly using a caliper. Tumor area was calculated and provided in Table 4 (see FIG. 7) and plotted in FIG. 8, graphically illustrating tumor size versus study day for Group 1, Group 2 and Group 3. For the in-life period graph, Days 0-49, the tumor size of animals that died or sacrificed during the in-life period was plotted as the last tumor measurement for the next of the in-life period.

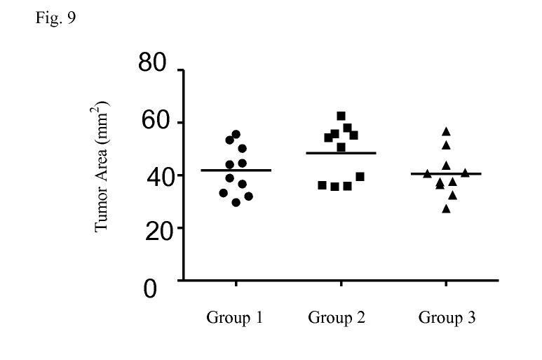

No difference in tumor size was observed prior to tumor removal (Day 7), see FIG. 9, which graphically illustrates tumor size in Group 1, Group 2 and Group 3. During the in-life period, (Days 14-28) no difference in tumor size was observed in mice treated with IL-2 only (Group 2) when compared with the untreated mice (Group 1). Mice treated for 21 days with propranolol and etodolac and IL-2 in Purastat (Group 3), exhibited a smaller tumor when compared to mice of Groups 1 and 2. ANOVA analysis of tumor size showed a significant decrease in tumor size when compared animals of Group 1 to Group 3 on Day 21 only. Due to the small number of animals that survived beyond Day 21, inferential statistics was not performed.

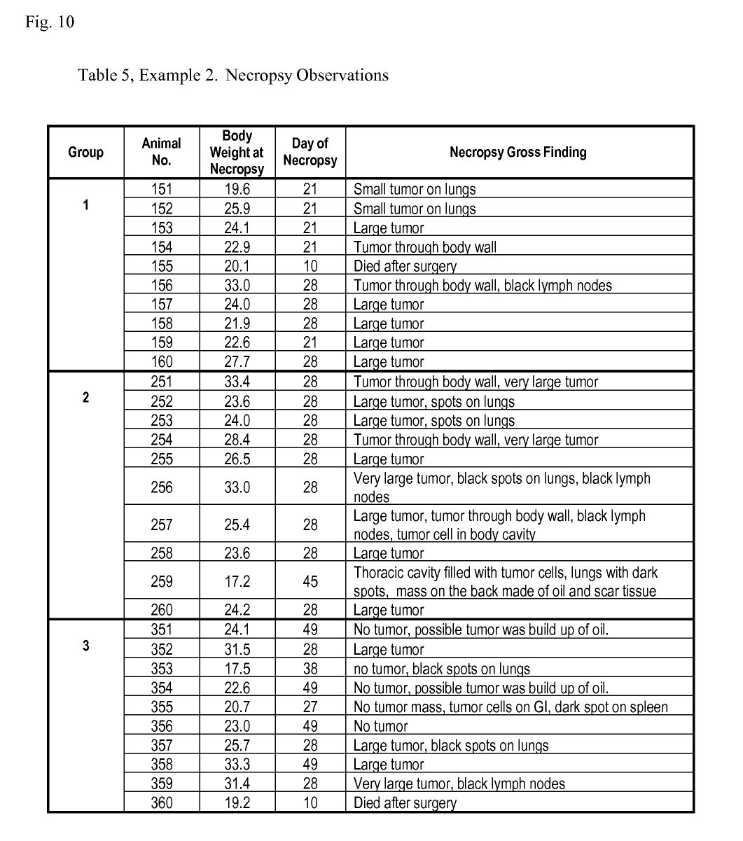

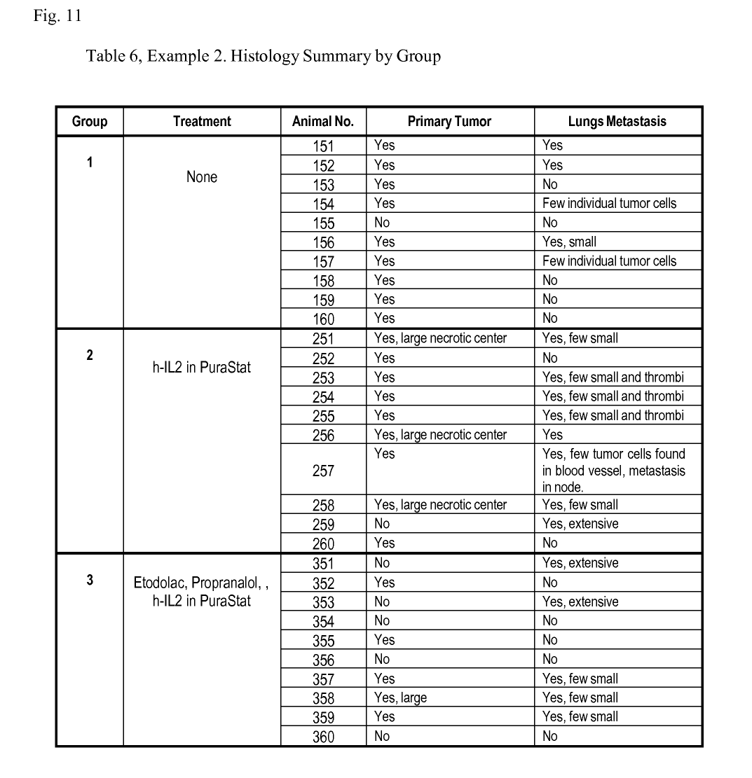

Necropsy: Necropsies were performed on all animals following death or moribund sacrifice. Major necropsy findings are provided in Table 5, FIG. 10. Gross necropsies confirmed that the mice developed solid tumors at the site of injection. In some animals the tumor metastasized into the abdomen and thoracic cavities, and some mice exhibited dark spots on lungs, bronchi, spleen, and kidneys. Two of four mice sacrifice on day 49, had no local or metastatic tumors.

Histology: Tumors and lungs from all animals were examined histopathologically. Fixed tissues were gross trimmed, processed through a graded series of alcohols, oriented and embedded in paraffin, microtome-sectioned at 3- to 5-μm thicknesses, slide-mounted, stained with hematoxylin & eosin (H&E), and cover-slipped by standard methodology. A histology summary is provided in Table 6, FIG. 11.

### Data Showing Synergy Statistically Significant

Group 3 (IL2+etodolac+propranolol) showed a statistical significant increase (p-value <0.05) in percent survival versus Group 1 with a p-value of 0.015 for day 35, a p-value of 0.041 for day 41 and a p-value of 0.041 for day 49.

Group 3 showed a statistically significant increase (p-value <0.05) in percent survival versus Group 2 with a p-value=0.033 for day 49 and showed a positive trend (p-value <0.20) with a p-value of 0.057 for day 35 and a p-value of 0.141 for day 41.

### Using a Fisher Exact Test for a 2×2 Contingency Table:

Statistical analysis: used Fisher Exact Test Calculator; this is a Fisher exact test calculator for a 2×2 contingency table. The Fisher exact test tends to be employed instead of Pearson's chi-square test when sample sizes are small.

The first stage is to enter group and category names in the textboxes below. Note: You can overwrite “Category 1”, “Category 2”, etc.; see e.g.: http://www.socscistatistics.com/tests/fisher/Default2.aspx

### SUMMARY—CONCLUSIONS

These data demonstrate that tumor removal followed by a single topical application of the exemplary composition provided herein comprising h-IL2 in (e.g., formulated in) a hydrogel material comprising a 16-amino acid synthetic peptide (Ac-[RADA]₄-CONH₂), e.g., a PURASTAT™ hydrogel, at a tumor site, e.g., a tumor excision site, along with daily administration of propranolol and etodolac (e.g., VT-122™) can reduce the tumor's size and protect against tumor growth and prevent metastasis; in the tested mice there were with no local tumor recurrences.

A number of aspects of the invention have been described. Nevertheless, it will be understood that various modifications may be made without departing from the spirit and scope of the invention. Accordingly, other aspects are within the scope of the following claims.

1. A product of manufacture, a device or a composition, comprising:
(a) a sterile hydrogel comprising a hydrogel material, wherein the hydrogel is:
(i) in a substantially liquid form capable of setting, gelling or self-assembling;
(ii) a partially assembled or gelled hydrogel, in a partially assembled or gelled form; or,
(iii) in a set, gelled or self-assembled state; or a substantially set, gelled or self-assembled state,
and optionally the set, gelled or self-assembled state is in situ; and
(b) one or a plurality of compositions or compounds comprising:
(i) a biologic, a drug or an immunostimulating agent or reagent,
(ii) an antigen or an immunogen, or a plurality of antigens or immunogens,
(iii)
(1) a biologic, a drug or an immunostimulating agent or reagent, and
(2) an antigen or an immunogen, or a plurality of antigens or immunogens,
(iv) an anticancer agent or reagent, or
(v) any combination of (i), (ii), (iii) and (iv), or all of (i) to (iv).

2. The product of manufacture, device or composition of claim 1, wherein:
(a) the sterile hydrogel material or sterile hydrogel is mixed with the one or the plurality of compositions or compounds of claim 1(b);
(b) the one or the plurality of compositions or compounds of claim 1(b) are first mixed in a sterile pure water or a sterile isotonic solution or buffer; or
(c) the one or the plurality of compositions or compounds of claim 1(b) are mixed with the sterile hydrogel material or sterile hydrogel:
(i) while the hydrogel is still in a substantially liquid state, un-self-assembled state, or ungelled state;
(ii) before the hydrogel has self-assembled, set or gelled,
(iii) before the hydrogel has set or self-assembled into a 3D hydrogel,
(iv) after the set, gelled or self-assembled hydrogel, or the substantially set, gelled or self-assembled hydrogel, has been disrupted or sheared; or
(v) at the same time the hydrogel has set, gelled or self-assembled hydrogel, or the hydrogel has substantially set, gelled or self-assembled.

3. The product of manufacture, device or composition of claim 1, wherein:
(a) the hydrogel is capable of self-assembling, gelling or setting when exposed to an environment comprising a salt concentrations ≧1 mM, or gelation, self-assembly or setting is initiated by salt concentrations ≧1 mM;
(b) the hydrogel is capable of self-assembling, gelling or setting into a 3D hydrogel having a nanometer scale and/or a fibrous structure with an average pore size of between about 50 to 200 nm; or
(c) the hydrogel is at a concentration of about: 0.1% to 5% (w/v), 0.5% to 4% (w/v), 1% to 3% (w/v), 1% to 10% (w/v), 1% to 15% (w/v), 1% to 20% (w/v), 1% to 25% (w/v), 1% to 30% (w/v), 1% to 40% (w/v), or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more (w/v).

4. The product of manufacture, device, or composition of claim 1, wherein:
(a) the hydrogel or hydrogel material comprises a self-assembling peptide;
(b) the hydrogel or hydrogel material comprises a plurality of synthetic peptides characterized by stable B-sheet structure with ionic side-chain interactions after setting, gelling or self-assembling;
(c) the hydrogel or hydrogel material comprises a 16-amino acid synthetic peptide (Ac-[RADA]₄-CONH₂), or SEQ ID NO:1, and optionally the hydrogel comprises PuraMatrix™ (PuraMatrix™) (BD Biosciences, San Jose, Calif.), or PuraDerm™ (PuraDerm™) (3DMatrix, Ltd, Tokyo, Japan);
(d) the hydrogel or hydrogel material comprises a self-assembling peptide comprising the sequence Lys-Leu-Asp-Leu-Lys-Leu-Asp-Leu-Lys-Leu-Asp-Leu (KLDL)₃ (SEQ ID NO:2);
(e) the hydrogel or hydrogel material comprises a self-assembling peptide comprising the sequence Ile-Glu-Ile-Lys-Ile-Glu-Ile-Lys-Ile-Glu-Ile-Lys-Ile (IEIK)₃I (SEQ ID NO:3);
(f) the hydrogel or hydrogel material comprises a cellulose, a chitin, a chitosan or a deacetylated chitin, a laminin, a collagen, an elastin, a fibrin, a gelatin, an alginic acid, a hyaluronic acid (HA), or a combination thereof,
wherein optionally the HA comprise a thiolated HA or a tyraminated HA;
or optionally the collagen comprises a collagen IV or a collagen I,
or optionally the cellulose comprises a hemicellulose methyl cellulose (MC), a hydroxypropyl cellulose (HPC), a hydroxypropylmethyl cellulose (HPMC), a carboxymethyl cellulose (CMC) or a cellulose-inorganic hybrid hydrogel;
(g) the hydrogel or hydrogel material comprises a polyethylene glycol (PEG), a polyethelene glycol diacrylate (PEGDA), an ethylene glycol dimethacrylate (EGDMA); a cyclodextrin; a p-dioxanone; a hydroxyethyl methacrylate; a poly(methyl methacrylate); a methylene-bis-acrylamide; a poly(acrylic acid); a polyacrylonitrile; a poly(butylene oxide); a polycaprolactone; a poly(ethylene imine); a poly(ethylene oxide); a poly(ethyl methacrylate); a propylene fumarate; a poly(glucosylethyl methacrylate); a poly(hydroxy butyrate); a poly(hydroxyethyl methacrylate); a poly(hydroxypropyl methacrylamide); a poly(lactic acid); a poly(lactic-co-glycolic acid); PNIPAAm, poly(N-isopropyl acrylamide); a poly(N-vinyl pyrrolidone); a poly(propylene oxide); a poly(vinyl alcohol); a poly(vinyl acetate); a poly(vinyl amine), or any combination thereof; or
(h) the hydrogel or hydrogel material comprises any combination of (a) to (g).

5. The product of manufacture, device or composition of claim 1, wherein
(a) the sterile pure water or a sterile isotonic solution or buffer comprises a saline, a phosphate buffered saline (PBS), or an equivalent buffer;
(b) the product of manufacture, device or composition of (a), wherein:
(1) the saline is used at an undiluted concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more; or
(2) the PBS is at a concentration of about 0.25% to 2.5%, 0.25% to 1.5%, 0.5% to 1.0%, 0.54%, 0.6%, 0.7%, 0.8%, 0.9% or 1.0%, or at a concentration of about: 0.1% to 5%, 0.5% to 4%, 1% to 3%, 1% to 10%, 1% to 15%, 1% to 20%, 1% to 25%, 1% to 30%, 1% to 40%, or about 0.1%, 0.25%, 0.5%, 0.75%, 1.0%, 1.5%, 2.0%, 2.5%, 3.0%, 3.5%, 4%, 5%, 10%, 15%, 20%, 25%, 30%, 35%, or 40% or more.

6. The product of manufacture, device or composition of claim 1, wherein the antigen, immunogen, or a plurality of antigens or immunogens, comprises:
(a) a synthetic, recombinant, partially purified, substantially purified or purified antigen, immunogen, or a plurality of antigens or immunogens, or any combination thereof;
(b) a small molecule or a biological molecule,
wherein optionally the biological molecule is or comprises a peptide, a polypeptide, a carbohydrate, a lipid, or any combination thereof,
and optionally the polypeptide comprises an antibody, or an anti-cancer or anti-tumor antibody,
and optionally the anti-cancer or anti-tumor antibody is an alemtuzumab, a brentuximab vedotin, a cetuximab, a gemtuzumab ozogamicin, an abritumomab tiuxetan, a nimotuzumab, an ofatumumab, a panitumumab, a rituximab, a tositumomab, or a trastuzumab;
(c) a cancer or tumor antigen, immunogen, or a plurality of antigens or immunogens, or any combination thereof;
(d) a cancer or a tumor cell extract, or a processed cancer or tumor cell,
wherein optionally the processed cancer or tumor cell is a minced cancer or tumor tissue or cell, and optionally the cancer or tumor tissue is minced with a device for making a mixed thickness skin micrograft or a split-thickness skin graft, or an XPANSION® device or an Xpansion Micrografting System® (SteadMed Medical, Fort Worth, Tex.),
and optionally the processed cancer or tumor cell is an irradiated cancer or tumor cell;
(e) the antigen, immunogen, or plurality of antigens or immunogens of any of (a) to (d), wherein the antigen, immunogen, or plurality of antigens or immunogens is mixed or treated with a cross-linking agent, a glutaraldehyde, a formaldehyde, a preservative, a neomycin, a polymyxin B, polihexanide, or any combination thereof, or the antigen, immunogen, or plurality of antigens or immunogens are irradiated; or
(f) any combination of (a) through (e).

7. The product of manufacture, device or composition of claim 1, wherein the biologic, the drug or the immunostimulating agent or reagent comprises:
(a) a cytokine,
wherein optionally the cytokine comprises an IL-2 or an interferon (IFN), and optionally the interferon is an alpha-IFN or a gamma-IFN;
and optionally the IL-2 is a recombinant IL-2, an aldesleukin, or a Proleukin (Prometheus Laboratories),
wherein optionally the IL-2, recombinant IL-2, or aldesleukin is dosages at about: 0.1 to 20, 1.0 to 20, 1 to 10, or 4 to 5, or 4.5 millions of IUs per cycle; or is dosaged for: 1 to 5, 2 to 4, or 3 cycles number of cycles of therapy;
(b) an immune checkpoint blockade agent, or an agent that blocks the interaction between a transmembrane programmed cell death 1 protein (PD-1; also known as CD279) and its ligand, PD-1 ligand 1 (PD-L1), or an ipilumumab (CTLA-4 mAb), or nivolumab (PD-1 mAb), or pembrolizumab (PD-1 mAb), or a lambrolizumab (a PD-L1 mAb);
(c) an activator of a pattern recognition receptor (PRR) or a toll-like receptor 7 (TLR7), or an imiquimod;
(d) chemotherapeutic agent,
wherein optionally the chemotherapeutic agent comprises a doxorubicin or a carboplatin, or comprises an inducer of apoptosis or a mitotic inhibitor or anti-microtubule inhibitor, or an alkylating agent, or a topoisomerase inhibitor, or a glycopeptide antibiotic, or steroid receptor inhibitor, or a matrix metalloproteinase (MMP) inhibitor, or an mTOR (mammalian target of rapamycin) inhibitor, or a macrolide or a composition comprising a macrolide ring, an aromatase inhibitor,
and optionally the inducer of apoptosis or a mitotic inhibitor or anti-microtubule inhibitor comprises or consists of a raltitrexed or equivalent, or Tomudex™; a doxorubicin or equivalent, or ADRIAMYCIN™; a fluorouracil or 5-fluorouracil or equivalent; a paclitaxel or equivalent, or TAXOL™ or ABRAXANE™; a docetaxel or equivalent, or TAXOTERE™; a larotaxel, tesetaxel or ortataxel or equivalent; an epothilone or an epothilone A, B, C, D, E or F or equivalent; an ixabepilone (also known as azaepothilone B) or equivalent, or BMS-247550™; a vincristine (also known as leurocristine) or equivalent, or Oncovin™; a vinblastin, vinblastine, vindesine, vinflunine, vinorelbine or Navelbine™ or equivalent; or, any combination thereof,
and optionally the alkylating agent comprises or consists of a cisplatin or equivalent; a cisplatinum or equivalent; a cis-diamminedichloridoplatinum(II) (CDDP) or equivalent; a carboplatin or equivalent; a oxaloplatin or equivalent; a cyclophosphamide (cytophosphane) or equivalent, or Endoxan™, Cytoxan™, Neosar™ or Revimmune™; a mechlorethamine or equivalent; a chlormethine or equivalent; a mustine or equivalent; a nitrogen mustard or equivalent; a chlorambucil or equivalent, or Leukeran™ or, a combination thereof,
and optionally the topoisomerase inhibitor comprises or consists of an etoposide or equivalent, or Eposin™, Etopophos™, Vepesid™ or VP-16™; an amsacrine or equivalent; a topotecan or equivalent, or Hycamtin™ a teniposide or equivalent, or Vumon™ or VM-26™; an epipodophyllotoxin or equivalent; a camptothecin or equivalent; an irinotecan or equivalent, or Camptosar™; or, a combination thereof,
and optionally the glycopeptide antibiotic comprises or consists of a bleomycin or equivalent or a bleomycin A2 or B2 or equivalent; a mitomycin or a mitomycin C or equivalent, a plicamycin (also known as mithramycin) or equivalent, or Mithracin™; or, a combination thereof,
and optionally the steroid receptor inhibitor comprises or consists of an estrogen receptor modulator (a SERM), and optionally the estrogen receptor modulator comprises or consists of a tamoxifen or equivalent, or Nolvadex™, Istubal™ or Valodex™, and optionally the steroid inhibitor or an anti-steroid comprises or consists of a finasteride or equivalent, or Proscar™, Propecia™, Fincar™, Finpecia™, Finax™, Finast™, Finara™, Finalo™, Prosteride™, Gefina™, Appecia™, Finasterid IVAX™, Finasterid or Alternova™,
and optionally the macrolide or composition comprising a macrolide ring comprises or consists of a clarithromycin or equivalent, or Biaxin™, Klaricid™, Klabax™, Claripen™, Claridar™, Fromilid™ or Clacid™; an azithromycin or equivalent, or ZITHROMAX™, Zitromax™ or Sumamed™; a dirithromycin or equivalent; an erythromycin or equivalent; a roxithromycin or equivalent, or Roxo™, Surlid™, Rulide™, Biaxsig™, Roxar™, Roximycin™ or Coroxin™; a telithromycin or equivalent or KETEK™; a josamycin or equivalent; a kitasamycin or equivalent; a midecamycin or equivalent; oleandomycin or equivalent; a roxithromycin or equivalent, or Roxo™, Surlid™, Rulide™, Biaxsig™, Roxar™, Roximycin™ or Coroxin™; a troleandomycin or equivalent; or a tylosin or equivalent; or, any combination thereof,
and optionally the aromatase inhibitor comprises: a 4-Hydroxyandrostenedione, a 1,4,6-Androstatrien-3,17-dione (ATD), or a 4-Androstene-3,6,17-trione (6-OXO);
(e) a radiotherapy enhancing agent;
(f) a combination of at least one beta adrenergic receptor antagonist and at least one non-steroidal anti-inflammatory drug (NSAID), or a propranolol and an etodolac, or VT-122™ (Vicus Therapeutics, Morristown, N.J.);
(g) an H₂-receptor antagonist (H₂RA),
wherein optionally the H₂-receptor antagonist comprises or consists of a cimetidine or equivalent, or Tagamet™, Tagamet HB™ or Tagamet HB200™; a ranitidine or equivalent, or TRITEC™ or ZANTAC™; a famotidine or equivalent, or Pepcidine™ or Pepcid™ a nizatidine or equivalent, or TAZAC™ or AXID™;
(h) a proton pump inhibitor (a PPI),
wherein optionally the proton pump inhibitor comprises or consists of a benzimidazole compound or structure, or an imidazopyridine compound or structure,
and optionally the imidazopyridine compound or structure comprises or consists of a zolpidem or equivalent, or Ambien™, Ambien CR™, Ivedal™, Nytamel™, Stilnoct™, Stilnox™, Zoldem™, Zolnod™ or Zolpihexal™; an alpidem (also called ananxyl) or equivalent; a saripidem or equivalent; necopidem or equivalent;
(i) a metformin, or an N,N-Dimethylimidodicarbonimidic diamide, or a Glucophage™, Fortamet™, Glumetza™ or Riomet™, or a quinoline, an aminoquinoline, e.g., a 4-aminoquinoline or an 8-Aminoquinoline, e.g., a chloroquine (or Aralen™), a hydroxychloroquine (or Plaquenil™) a quinacrine (Atabrine™), a primaquine, a tafenoquine, or equivalents thereof; or
(j) any combination of (a) to (i).

8. The product of manufacture, device or composition of claim 1, wherein the anticancer agent or reagent comprises a radioactive particle or isotope; or a microscopic, radioactive glass microsphere; a plurality of radioactive glass microspheres, optionally about 20 to 30 micrometers in diameter; or, a TheraSphere.

9. The product of manufacture, device or composition of claim 1, wherein the anticancer agent or reagent comprises a drug-eluting or a cancer drug-eluting particle, liposome or bead, or a doxorubicin-loaded drug-eluting bead.

10. The product of manufacture, device or composition of claim 1, wherein the anticancer agent or reagent comprises:
a sorafenib or equivalent, or Nexavar™; a sunitinib or equivalent, or SUTENT™; an erlotinib or equivalent, or Tarceva™; an imatinib or equivalent, or GLEEVEC™; a lapatinib or equivalent, or Tykerb™; a toceranib or equivalent, or Palladia™; a masitinib or equivalent, or MASIVET™; a bevacizumab or equivalent, or Avastin™; a trastuzumab or equivalent, or HERCEPTIN™; a cetuximab or equivalent, or Erbitux™ a bevacizumab or equivalent, or Avastin™ or BIBW 2992; a gefitinib or equivalent, or Iressa™; a ranibizumab or equivalent, or LUCENTIS™; a pegaptanib or equivalent, or MACUGEN™; a dasatinib or equivalent, or BMS-354825™; a sunitinib or equivalent, or SUTENT™; a pazopanib or equivalent; a nilotinib or equivalent, or Tasigna™; a panitumumab or equivalent, or Vectibix™; a bandetinib or equivalent; a brivanib or equivalent, or E7080™; a thalidomide or equivalent, or THALOMID™; lenalidomide or equivalent, or Revlimid™; a bortezomib or equivalent, or VELCADE™ disulfiram or equivalent, or Antabuse™ or Antabus™ or an epigallocatechin gallate (EGCG) or equivalent; a demecolcine, an etoglucid or elsamitrucin, a lonidamine, a lucanthone, a mitotane or a mitoguazone or equivalent; or any combination thereof.

11. (canceled)

12. The product of manufacture, device or composition of claim 1, further comprising:
(a) an adjuvant;
(b) an immunostimulating cytokine or biologic; or
(f) any combination of (a) or (b).

13. A product of manufacture, device or composition of claim 1, wherein the product of manufacture, device or composition is in situ.

14. A device, a medical device, an implant, a breast implant, a prosthesis, a stent, a catheter, comprising a product of manufacture, device or composition of claim 1.

15. A method for:
(a) (i) treating, preventing or ameliorating a tumor or a cancer,
(ii) vaccinating or immunizing an individual against an antigen or an immunogen,
(iii) vaccinating or immunizing an individual against a cancer or tumor antigen or immunogen, or a plurality of antigens or immunogens, or any combination thereof,
(iv) immunostimulating an individual, or
(v) any combination of (i) to (iv),
comprising:
applying or administering to an individual in need thereof; or, applying or administering to a target cancer, tumor, tissue or organ, or an affected tissue or organ:
the product of manufacture, device or composition of claim 1;
(b) the method of (a), further comprising applying or administering to the individual in need thereof; or, applying or administering to the target cancer, tumor, tissue or organ, or affected tissue or organ, the product of manufacture, device or composition, or the device, medical device, implant, breast implant, prosthesis, stent or catheter, simultaneous with, in conjunction with, before and/or after a systemic therapy,
wherein optionally the product of manufacture, device or composition, or the device, medical device, implant, breast implant, prosthesis, stent or catheter is or are administered before the systemic therapy, or both are administered consecutively, or the product of manufacture, device or composition, or the device, medical device, implant, breast implant, prosthesis, stent or catheter is administered after the systemic therapy, or any combination thereof;
(c) the method of (b), wherein the systemic therapy comprises:
(i) treating, preventing or ameliorating a tumor or a cancer,
(ii) vaccinating or immunizing an individual against an antigen or an immunogen,
(iii) vaccinating or immunizing an individual against a cancer or tumor antigen or immunogen, or a plurality of antigens or immunogens, or any combination thereof,
(iv) immunostimulating an individual,
(v) providing an anticancer or antitumor treatment, or
(vi) any combination of (i) to (v);
(d) the method of (b) or (c), wherein the systemic therapy comprises a systemic anti-cancer or anti-tumor treatment, or an anti-cancer or anti-tumor immunotherapy or vaccination, or an anti-cancer or anti-tumor immunostimulation;
(e) the method of (d), wherein the systemic anti-cancer or anti-tumor treatment comprises administration, application, or use of a drug, a biologic, a nutrient, an anti-cancer or anti-tumor dietary regimen, a radioactive agent, a tumor ablative agent, or a combination thereof;
(f) the method of (d), wherein the systemic anti-cancer or anti-tumor treatment comprises administration, application, or use of an anti-cancer or anti-tumor radiotherapy or a proton beam therapy;
(g) the method of (d), wherein the systemic anti-cancer or anti-tumor treatment comprises administration, application, or use of a proton pump inhibitor (a PPI),
wherein optionally the proton pump inhibitor comprises or consists of a benzimidazole compound or structure, or an imidazopyridine compound or structure,
and optionally the imidazopyridine compound or structure comprises or consists of a zolpidem or equivalent, or Ambien™, Ambien CR™, Ivedal™, Nytamel™ Stilnoct™, Stilnox™, Zoldem™, Zolnod™ or Zolpihexal™; an alpidem (also called ananxyl) or equivalent; a saripidem or equivalent; necopidem or equivalent;
(h) the method of (d), wherein the systemic anti-cancer or anti-tumor treatment comprises administration, application, or use of an H₂-receptor antagonist (H₂RA),
wherein optionally the H₂-receptor antagonist comprises or consists of a cimetidine or equivalent, or Tagamet™, Tagamet HB™ or Tagamet HB200™; a ranitidine or equivalent, or TRITEC™ or ZANTAC™; a famotidine or equivalent, or Pepcidine™ or Pepcid™ a nizatidine or equivalent, or TAZAC™ or AXID™;
(i) the method of (d), wherein the systemic anti-cancer or anti-tumor treatment comprises administration, application, or use of a combination of at least one beta adrenergic receptor antagonist and at least one non-steroidal anti-inflammatory drug (NSAID), or a propranolol and an etodolac, or a VT-122™ (Vicus Therapeutics, Morristown, N.J.);
(j) the method of any of (d), wherein the systemic anti-cancer or anti-tumor treatment comprises administration, application, or use of a cytokine,
wherein optionally the cytokine comprises an IL-2 or an interferon (IFN),
and optionally the interferon is an alpha-IFN or a gamma-IFN;
and optionally the IL-2 is a recombinant IL-2, an aldesleukin, or a Proleukin (Prometheus Laboratories),
wherein optionally the IL-2, recombinant IL-2, or aldesleukin is dosages at about: 0.1 to 20, 1.0 to 20, 1 to 10, or 4 to 5, or 4.5 millions of IUs per cycle; or is dosaged for: 1 to 5, 2 to 4, or 3 cycles number of cycles of therapy;
(k) the method of any of (d), wherein the systemic anti-cancer or anti-tumor treatment comprises administration, application, or use of a an immune checkpoint blockade agent, or an agent that blocks the interaction between a transmembrane programmed cell death 1 protein (PD-1; also known as CD279) and its ligand, PD-1 ligand 1 (PD-L1), or an ipilumumab (CTLA-4 mAb) or nivolumab (PD-1 mAb), or pembrolizumab (PD-1 mAb), or a lambrolizumab (a PD-L1 mAb);
(l) the method of any of (d), wherein the systemic anti-cancer or anti-tumor treatment comprises administration, application, or use of an activator of a pattern recognition receptor (PRR) or a toll-like receptor 7 (TLR7), or an imiquimod;
(m) the method of any of (d), wherein the systemic anti-cancer or anti-tumor treatment comprises administration, application, or use of a radiotherapy enhancing agent;
(n) the method of any of (d), wherein the systemic anti-cancer or anti-tumor treatment comprises administration, application, or use of chemotherapeutic agent,
wherein optionally the chemotherapeutic agent comprises a doxorubicin or a carboplatin, or comprises an inducer of apoptosis or a mitotic inhibitor or anti-microtubule inhibitor, or an alkylating agent, or a topoisomerase inhibitor, or a glycopeptide antibiotic, or steroid receptor inhibitor, or a matrix metalloproteinase (MMP) inhibitor, or an mTOR (mammalian target of rapamycin) inhibitor, or a macrolide or a composition comprising a macrolide ring, an aromatase inhibitor;
(o) the method of any of (a) to (n), wherein the systemic anti-cancer or anti-tumor treatment comprises administration, application, or use of:
(1) a systemic immunotherapy,
(2) a combination of at least one beta adrenergic receptor antagonist and at least one non-steroidal anti-inflammatory drug (NSAID), or a propranolol and an etodolac, or a VT-122™,
(3) a proton pump inhibitor (a PPI), and
(4) an H₂-receptor antagonist (H₂RA);
(p) the method of any of (a) to (o), wherein the systemic anti-cancer or anti-tumor treatment comprises administration, application, or use of a chemotherapy and/or a radiotherapy, and use of a combination of at least one beta adrenergic receptor antagonist and at least one non-steroidal anti-inflammatory drug (NSAID), or a propranolol and an etodolac, or a VT-122™; or
(q) a method comprising any combination of (a) to (p).

16. A method for treating, preventing or ameliorating a tumor or a cancer, comprising:
(a) applying or administering to an individual in need thereof; or, applying or administering to an effected tissue; the product of manufacture, device or composition on of claim 1; and
(b) administering to the individual in need thereof:
(i) a systemic anti-cancer or anti-tumor treatment,
wherein optionally the systemic anti-cancer or anti-tumor treatment comprises administration of a drug, a biologic, a cytokine, a nutrient, an anti-cancer or anti-tumor dietary regimen, a radioactive agent, a tumor ablative agent, or
(ii) an anti-cancer or anti-tumor radiotherapy or a proton beam therapy,
wherein the anti-cancer or anti-tumor treatment of (a) is administered before the anti-cancer or anti-tumor treatment of (b), or both are administered consecutively, or the anti-cancer or anti-tumor treatment of (a) is administered after the anti-cancer or anti-tumor treatment of (b), or any combination thereof.

17. The method of claim 15, wherein the cancer or tumor is: a mastocytoma or a mast cell tumor, an ovarian cancer, pancreatic cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer.

18. The method of claim 16, wherein:
(a) (i) for step 16(a):
the product of manufacture, device or composition of claim 1; comprises:
(1) a combination of at least one beta adrenergic receptor antagonist and at least one non-steroidal anti-inflammatory drug (NSAID), or a propranolol and an etodolac, or a VT-122™ (Vicus Therapeutics, Morristown, N.J.),
wherein optionally the at least one beta adrenergic receptor antagonist and/or the at least one non-steroidal anti-inflammatory drug (NSAID) is/are administered locally or systemically, but separately from the product of manufacture, device or composition as set forth in any of claims 1 to 12; or, the device, medical device, implant, breast implant, prosthesis, stent or catheter of claim 14,
(2) a cytokine,
wherein optionally the cytokine comprises an IL-2 or an interferon (IFN),
and optionally the interferon is an alpha-IFN or a gamma-IFN;
and optionally the IL-2 is a recombinant IL-2, an aldesleukin, or a Proleukin (Prometheus Laboratories),
wherein optionally the IL-2, recombinant IL-2, or aldesleukin is dosages at about: 0.1 to 20, 1.0 to 20, 1 to 10, or 4 to 5, or 4.5 millions of IUs per cycle; or is dosaged for: 1 to 5, 2 to 4, or 3 cycles number of cycles of therapy, or
wherein optionally the cytokine is administered locally, but separately from the product of manufacture, device or composition as set forth in any of claims 1 to 12; or, the device, medical device, implant, breast implant, prosthesis, stent or catheter of claim 14,
(3) a combination of (1) and (2); or
(ii) for step 16(b), the systemic anti-cancer or anti-tumor treatment comprises an anti-cancer or anti-tumor radiotherapy or a proton beam therapy;
(b) the at least one beta adrenergic receptor antagonist and at least one non-steroidal anti-inflammatory drug (NSAID), are administered systemically, and the cytokine, optionally IL-2, is administered with (or as part of) the product of manufacture, device or composition as set forth in any of claims 1 to 12; or, the device, medical device, implant, breast implant, prosthesis, stent or catheter of claim 14; or
(c) the method of (a) or (b), wherein the cancer being treated, prevented or ameliorated is a mast cell tumor or a melanoma.

19. A kit, or an integrated point of care mixing kit, comprising
(a) the product of manufacture, device or composition as of claim 1,
wherein optionally the sterile hydrogel material or sterile hydrogel is: (i) in a substantially liquid form capable of setting, gelling or self-assembling; (ii) a partially assembled or gelled hydrogel; or, (iii) in a set, gelled or self-assembled state; or a substantially set, gelled or self-assembled state;
(b) the of (a), kit further comprising instructions for practicing any of the methods of claims 15 to 18.

20. A therapeutic combination comprising:
(a) (1)
a product of manufacture, device, or composition of claim 1; and
(2)
(i) a biologic, a drug or an immunostimulating agent or reagent,
(ii) an antigen or an immunogen, or a plurality of antigens or immunogens,
(iii) a biologic, a drug or an immunostimulating agent or reagent, and an antigen or an immunogen, or a plurality of antigens or immunogens,
(iv) an anticancer agent or reagent, or
(v) any combination thereof;
(b) the therapeutic combination of (a), wherein the composition or compositions, or the biologics, drugs, immunostimulating agents or reagents, antigens or immunogens, or anticancer agents or reagents, of (a), are systemically administered;
(c) the therapeutic combination of (a) or (b), wherein the composition or compositions, or the biologics, drugs, immunostimulating agents or reagents, antigens or immunogens, or anticancer agents or reagents, of (a), comprise:
a combination of at least one beta adrenergic receptor antagonist and at least one non-steroidal anti-inflammatory drug (NSAID), or
a propranolol and an etodolac, or
a VT-122™ (Vicus Therapeutics, Morristown, N.J.);
(d) the therapeutic combination of any of (a) to (c), wherein the therapeutic combination is used in the treatment, amelioration or healing of: a cancer or a tumor;
(e) the therapeutic combination of (d), wherein the cancer or tumor is: a mastocytoma or a mast cell tumor, an ovarian cancer, pancreatic cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a Histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer.

21-31. (canceled)


SEQUENCE LISTING (3 sequences)

SEQ ID NO: 1 - protein, 16 aa, artificial sequence
  synthetic peptide
RADARADARADARADA

SEQ ID NO: 2 - protein, 12 aa, artificial sequence
  synthetic peptide
KLDLKLDLKLDL

SEQ ID NO: 3 - protein, 13 aa, artificial sequence
  synthetic peptide
IEIKIEIKIEIKI